SEQUENCE LISTING (71 sequences)

SEQ ID NO: 1 - protein, 504 aa, Homo sapiens
FSAPSRISAWFGPPASTPASTMSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 2 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTLLIKTVETRDGQVINETSQHHDDLE

SEQ ID NO: 3 - protein, 338 aa, Homo sapiens [sequence omitted]

SEQ ID NO: 4 - protein, 257 aa, Homo sapiens
MDAIKKKMQMLKLDKENAIDRAEQAEADKKQAEDRCKQLEEEQQALQKKLKGTEDEVEKYSESVKEAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESRARQLEEELRTMDQALKSLMASEEEYSTKEDKYEEEIKLLEEKLKEAETRAEFAERSVAKLEKTIDDLE

SEQ ID NO: 5 - protein, 263 aa, Homo sapiens
KANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 6 - protein, 84 aa, Homo sapiens
VKQIESKTAFQEALDAAGDKLVVVDFSATWCGPCKMIKPFFHDVASECEVKCMPTFQFFKKGQKVGEFSGANKEKLEATINELV

SEQ ID NO: 7 - protein, 248 aa, Homo sapiens
APSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

SEQ ID NO: 8 - protein, 339 aa, Homo sapiens [sequence omitted]

SEQ ID NO: 9 - protein, 237 aa, Homo sapiens
MAGLNSLEAVKRKIQALQQQADEAEDRAQGLQRELDGERERREKAEGDVAALNRRIQLVEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEVYRRKHQELQAMQMEL

SEQ ID NO: 10 - protein, 105 aa, Homo sapiens
MCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 11 - protein, 116 aa, Homo sapiens
MRALGQNPTNAEVLKVLGNPKSDEMNVKVLDFEHFLPMLQTVAKNKDQGTYEDYVEGLRVFDKEGNGTVMGAEIRHVLVTLGEKMTEEEVEMLVAGHEDSNGCINYEAFVRHILSG

SEQ ID NO: 12 - protein, 469 aa, Homo sapiens
MSQAYSSSQRVSSYRRTFGGAPGFPLGSPLSSPVFPRAGFGSKGSSSSVTSRVYQVSRTSGGAGGLGSLRASRLGTTRTPSSYGAGELLDFSLADAVNQEFLTTRTNEKVELQELNDRFANYIEKVRFLEQQNALAAEVNRLKGREPTRVAELYEEELRELRRQVEVLTNQRARVDVERDNLLDDLQRLKAKLQEEIQLKEEAENNLAAFRADVDAATLARIDLERRIESLNEEIAFLKKVHEEEIRELQAQLQEQQVQVEMDMSKPDLTAALRDIRAQYETIAAKNISEAEEWYKSKVSDLTQAANKNNDALRQAKQEMMEYRHQIQSYTCEIDALKGTNDSLMRQMRELEDRFASEASGYQDNIARLEEEIRHLKDEMARHLREYQDLLNVKMALDVEIATYRKLLEGEESRINLPIQTYSALNFRETSPEQRGSEVHTKKTVMIKTIETRDGEVVSEATQQQHEVL

SEQ ID NO: 13 - protein, 893 aa, Homo sapiens
MATEEFIIRIPPYHYIHVLDQNSNVSRVEVGPKTYIRQDNERVLFAPMRMVTVPPRHYCTVANPVSRDAQGLVLFDVTGQVRLRHADLEIRLAQDPFPLYPGEVLEKDITPLQVVLPNTALHLKALLDFEDKDGDKVVAGDEWLFEGPGTYIPRKEVEVVEIIQATIIRQNQALRLRARKECWDRDGKERVTGEEWLVTTVGAYLPAVFEEVLDLVDAVILTEKTALHLRARRNFRDFRGVSRRTGEEWLVTVQDTEAHVPDVHEEVLGVVPITTLGPHNYCVILDPVGPDGKNQLGQKRVVKGEKSFFLQPGEQLEQGIQDVYVLSEQQGLLLRALQPLEEGEDEEKVSHQAGDHWLIRGPLEYVPSAKVEVVEERQAIPLDENEGIYVQDVKTGKVRAVIGSTYMLTQDEVLWEKELPPGVEELLNKGQDPLADRGEKDTAKSLQPLAPRNKTRVVSYRVPHNAAVQVYDYREKRARVVFGPELVSLGPEEQFTVLSLSAGRPKRPHARRALCLLLGPDFFTDVITIETADHARLQLQLAYNWHFEVNDRKDPQETAKLFSVPDFVGDACKAIASRVRGAVASVTFDDFHKNSARIIRTAVFGFETSEAKGPDGMALPRPRDQAVFPQNGLVVSSVDVQSVEPVDQRTRDALQRSVQLAIEITTNSQEAAAKHEAQRLEQEARGRLERQKILDQSEAEKARKELLELEALSMAVESTGTAKAEAESRAEAARIEGEGSVLQAKLKAQALAIETEAELQRVQKVRELELVYARAQLELEVSKAQQLAEVEVKKFKQMTEAIGPSTIRDLAVAGPEMQVKLLQSLGLKSTLITDGSTPINLFNTAFGLLGMGPEGQPLGRRVASGPSPGEGISPQSAQAPQAPGDNHVVPVLR

SEQ ID NO: 14 - protein, 371 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGGGPGYSGGSRGYGSGGQGYGNQGSGYGGSGSYDSYNNGGGGGFGGGSGSNFGGGGSYNDFGNYNNQSSNFGPMKGGNFGGRSSGPYGGGGQYFAKPRNQGGYGGSSSSSSYGSGRR

SEQ ID NO: 15 - protein, 96 aa, Homo sapiens
PSQMEHAMETMMFTFHKFAGDKGYLTKEDLRVLMEKEFPGFLENQKDPLAVDKIMKDLDQCRDGKVGFQSFFSLIAGLTIACNDYFVVHMKQKGKK

SEQ ID NO: 16 - protein, 361 aa, Homo sapiens
MITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 17 - protein, 172 aa, Homo sapiens [sequence omitted]

SEQ ID NO: 18 - protein, 284 aa, Homo sapiens
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDRYEEEIKVLSDKLKEAETRAEFAERSVTKLEKSIDDLEEKVAHAKEENLSMHQMLDQTLLELNNM

SEQ ID NO: 19 - protein, 284 aa, Homo sapiens
MDAIKKKMQMLKLDKENAIDRAEQAEADKKQAEDRCKQLEEEQQALQKKLKGTEDEVEKYSESVKEAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESRARQLEEELRTMDQALKSLMASEEEYSTKEDKYEEEIKLLEEKLKEAETRAEFAERSVAKLEKTIDDLEETLASAKEENVEIHQTLDQTLLELNNL

SEQ ID NO: 20 - protein, 171 aa, Homo sapiens
MSSKRTKTKTKKRPQRATSNVFAMFDQSQIQEFKEAFNMIDQNRNGFIDKEDLHDMLASLGKNPTDEYLDAMMNEAPGPINFTMFLTMFGEKLNGTDPEDVIRNAFACFDEEATGTIQEDYLRELLTTMGDRFTDEEVDELYREAPIDKKGNFNYIEFTRILKHGAKDKDD

SEQ ID NO: 21 - protein, 141 aa, Homo sapiens
VLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 22 - protein, 248 aa, Homo sapiens
MAGLNSLEAVKRKIQALQQQADEAEDRAQGLQRELDGERERREKAEGDVAALNRRIQLFEEELDRAQERLATALQKLEEAEKAADESERGMKVIENRAMKDEEKMEIQEMQLKEAKHIAEEADRKYEEVARKLVILEGELERAEERAEVSELKCGDLEEELKNVTNNLKSLEAASEKYSEKEDKYEEEIKLLSDKLKEAETRAEFAERTVAKLEKTIDDLEEKLAQAKEENVGLHQTLDQTLNELNCI

SEQ ID NO: 23 - protein, 201 aa, Homo sapiens
MANKGPSYGMSREVQSKIEKKYDEELEERLVEWIIVQCGPDVGRPDRGRLGFQVWLKNGVILSKLVNSLYPDGSKPVKVPENPPSMVFKQMEQVAQFLKAAEDYGVIKTDMFQTVDLFEGKDMAAVQRTLMALGSLAVTKNDGHYRGDPNWFMKKAQEHKREFTESQLQEGKHVIGLQMGSNRGASQAGMTGYGRPRQIIS

SEQ ID NO: 24 - protein, 469 aa, Homo sapiens
MSIHFSSPVFTSRSAAFSGRGAQVRLSSARPGGLGSSSLYGLGASRPRVAVRSAYGGPVGAGIREVTINQSLLAPLRLDADPSLQRVRQEESEQIKTLNNKFASFIDKVRFLEQQNKLLETKWTLLQEQKSAKSSRLPDIFEAQIAGLRGQLEALQVDGGRLEAELRSMQDVVEDFKNKYEDEINRRTAAENEFVVLKKDVDAAYMSKVELEAKVDALNDEINFLRTLNETELTELQSQISDTSVVLSMDNSRSLDLDGIIAEVKAQYEEMAKCSRAEAEAWYQTKFETLQAQAGKHGDDLRNTRNEISEMNRAIQRLQAEIDNIKNQRAKLEAAIAEAEERGELALKDARAKQEELEAALQRAKQDMARQLREYQELMSVKLALDIEIATYRKLLEGEESRLAGDGVGAVNISVMNSTGGSSSGGGIGLTLGGTMGSNALSFSSSAGPGLLKAYSIRTASASRRSARD

SEQ ID NO: 25 - protein, 360 aa, Homo sapiens
MCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANLEKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 26 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAMFHRKLFEELVRASSHSTDLMEAMAMGSVEASYKCLAAALIVLTESGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 27 - protein, 154 aa, Homo sapiens
MSKNTVSSARFRKVDVDEYDENKFVDEEDGGDGQAGPDEGEVDSCLRHSITGNMTAALQAALKNPPINTKSQAVKDRAGSIVLKVLISFKANDIEKAVQSLDKNGVDLLMKYIYKGFESPSDNSSAMLLQWHEKALAAGGVGSIVRVLTARKTV

SEQ ID NO: 28 - protein, 175 aa, Homo sapiens
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 29 - protein, 248 aa, Homo sapiens [sequence omitted]

SEQ ID NO: 30 - protein, 142 aa, Homo sapiens [sequence omitted]

SEQ ID NO: 31 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 32 - protein, 199 aa, Homo sapiens
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 33 - protein, 501 aa, Homo sapiens
MSSSGTPDLPVLLTDLKIQYTKIFINNEWHDSVSGKKFPVFNPATEEELCQVEEGDKEDVDKAVKAARQAFQIGSPWRTMDASERGRLLYKLADLIERDRLLLATMESMNGGKLYSNAYLSDLAGCIKTLRYCAGWADKIQGRTIPIDGNFFTYTRHEPIGVCGQIIPWNFPLVMLIWKIGPALSCGNTVVVKPAEQTPLTALHVASLIKEAGFPPGVVNIVPGYGPTAGAAISSHMDIDKVAFTGSTEVGKLIKEAAGKSNLKRVTLELGGKSPCIVLADADLDNAVEFAHHGVFYHQGQCCIAASRIFVEESIYDEFVRRSVERAKKYILGNPLTPGVTQGPQIDKEQYDKILDLIESGKKEGAKLECGGGPWGNKGYFVQPTVFSNVTDEMRIAKEEIFGPVQQIMKFKSLDDVIKRANNTFYGLSAGVFTKDIDKAITISSALQAGTVWVNCYGVVSAQCPFGGFKMSGNGRELGEYGFHEYTEVKTVTVKISQKNS

SEQ ID NO: 34 - protein, 284 aa, Homo sapiens
MDAIKKKMQMLKLDKENALDRAEQAEADKKAAEDRSKQLEEDIAAKEKLLRVSEDERDRVLEELHKAEDSLLAAEEAAAKAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNDLKSLEAQAEKYSQKEDRYEEEIKVLSDKLKEAETRAEFAERSVTKLEKSIDDLEDELYAQKLKYKAISEELDHALNDMTSI

SEQ ID NO: 35 - protein, 323 aa, Homo sapiens
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 36 - protein, 147 aa, Homo sapiens
MVHLTPEEKTAVNALWGKVNVDAVGGEALGRLLVVYPWTQRFFESFGDLSSPDAVMGNPKVKAHGKKVLGAFSDGLAHLDNLKGTFSQLSELHCDKLHVDPENFRLLGNVLVCVLARNFGKEFTPQMQAAYQKVVAGVANALAHKYH

SEQ ID NO: 37 - protein, 609 aa, Homo sapiens [sequence omitted]

SEQ ID NO: 38 - protein, 321 aa, Homo sapiens [sequence omitted]

SEQ ID NO: 39 - protein, 104 aa, Homo sapiens
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQ

SEQ ID NO: 40 - protein, 172 aa, Homo sapiens [sequence omitted]

SEQ ID NO: 41 - protein, 501 aa, Homo sapiens; identical to SEQ ID NO: 33

SEQ ID NO: 42 - protein, 501 aa, Homo sapiens
MSSSGTPDLPVLLTDLKIQYTKIFINNEWHDSVSGKKFPVFNPATEEELCQVEEGDKEDVDKAVKAARQAFQIGSPWRTMDASERGRLLYKLADLIERDRLLLATMESMNGGKLYSNAYLNDLAGCIKTLRYCAGWADKIQGRTIPIDGNFFTYTRHEPIGVCGQIIPWNFPLVMLIWKIGPALSCGNTVVVKPAEQTPLTALHVASLIKEAGFPPGVVNIVPGYGPTAGAAISSHMDIDKVAFTGSTEVGKLIKEAAGKSNLKRVTLELGGKSPCIVLADADLDNAVEFAHHGVFYHQGQCCIAASRIFVEESIYDEFVRRSVERAKKYILGNPLTPGVTQGPQIDKEQYDKILDLIESGKKEGAKLECGGGPWGNKGYFVQPTVFSNVTDEMRIAKEEIFGPVQQIMKFKSLDDVIKRANNTFYGLSAGVFTKDIDKAITISSALQAGTVWVNCYGVVSAQCPFGGFKMSGNGRELGEYGFHEYTEVKTVTVKISQKNS

SEQ ID NO: 43 - protein, 534 aa, Homo sapiens
ASLSLAPVNIFKAGADEERAETARLTSFIGAIAIGDLVKSTLGPKGMDKILLSSGRDASLMVTNDGATILKNIGVDNPAAKVLVDMSRVQDDEVGDGTTSVTVLAAELLREAESLIAKKIHPQTIIAGWREATKAAREALLSSAVDHGSDEVKFRQDLMNIAGTTLSSKLLTHHKDHFTKLAVEAVLRLKGSGNLEAIHIIKKLGGSLADSYLDEGFLLDKKIGVNQPKRIENAKILIANTGMDTDKIKIFGSRVRVDSTAKVAEIEHAEKEKMKEKVERILKHGINCFINRQLIYNYPEQLFGAAGVMAIEHADFAGVERLALVTGGEIASTFDHPELVKLGSCKLIEEVMIGEDKLIHFSGVALGEACTIVLRGATQQILDEAERSLHDALCVLAQTVKDSRTVYGGGCSEMLMAHAVTQLANRTPGKEAVAMESYAKALRMLPTIIADNAGYDSADLVAQLRAAHSEGNTTAGLDMREGTIGDMAILGITESFQVKRQVLLSAAEAAEVILRVDNIIKAAPRKRVPDHHPC

SEQ ID NO: 44 - protein, 396 aa, Homo sapiens
MFLKAVVLTLALVAVAGARAEVSADQVATVMWDYFSQLSNNAKEAVEHLQKSELTQQLNALFQDKLGEVNTYAGDLQKKLVPFATELHERLAKDSEKLKEEIGKELEELRARLLPHANEVSQKIGDNLRELQQRLEPYADQLRTQVNTQAEQLRRQLDPLAQRMERVLRENADSLQASLRPHADELKAKIDQNVEELKGRLTPYADEFKVKIDQTVEELRRSLAPYAQDTQEKLNHQLEGLTFQMKKNAEELKARISASAEELRQRLAPLAEDVRGNLKGNTEGLQKSLAELGGHLDQQVEEFRRRVEPYGENFNKALVQQMEQLRQKLGPHAGDVEGHLSFLEKDLRDKVNSFFSTFKEKESQDKTLSLPELEQQQEQQQEQQQEQVQMLAPLES

SEQ ID NO: 45 - protein, 338 aa, Homo sapiens [sequence omitted]

SEQ ID NO: 46 - protein, 282 aa, Homo sapiens
AVPPTYADLGKSARDVFTKGYGFGLIKLDLKTKSENGLEFTSSGSANTETTKVTGSLETKYRWTEYGLTFTEKWNTDNTLGTEITVEDQLARGLKLTFDSSFSPNTGKKNAKIKTGYKREHINLGCDMDFDIAGPSIRGALVLGYEGWLAGYQMNFETAKSRVTQSNFAVGYKTDEFQLHTNVNDGTEFGGSIYQKVNKKLETAVNLAWTAGNSNTRFGIAAKYQIDPDACFSAKVNNSSLIGLGYTQTLKPGIKLTLSALLDGKNVNAGGHKLGLGLEFQA

SEQ ID NO: 47 - protein, 335 aa, Homo sapiens [sequence omitted]

SEQ ID NO: 48 - protein, 331 aa, Homo sapiens [sequence omitted]

SEQ ID NO: 49 - protein, 335 aa, Homo sapiens [sequence omitted]

SEQ ID NO: 50 - protein, 247 aa, Homo sapiens
MLVLRSALTRALASRTLAPQMCSSFATGPRQYDGIFYEFRSYYLKPSKMNEFLENFEKNAHLRTAHSELVGYWSVEFGGRMNTVFHIWKYDNFAHRTEVRKALAKDKEWQEQFLIPNLALIDKQESEITYLVPWCKLEKPPKEGVYELATFQMKPGGPALWGDAFKRAVHAHVNLGYTKLVGVFHTEYGALNRVHVLWWNESADSRAAGRHKSHEDPRVVAAVRESVNYLVSQQNMLLIPTSFSPLK

SEQ ID NO: 51 - protein, 147 aa, Homo sapiens
MASGNARIGKPAPDFKATAVVDGAFKEVKLSDYKGINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 52 - protein, 198 aa, Homo sapiens
MASGNARIGKPAPDFKATAVVDGAFKEVKLSDYKGKYVVLFFYPLDFTFVCPTEIIAFTTVKRTSAKLGCEVLGVSVDSQFTHLAWINTPRKEGGLGPLNIPLLADVTRRLSEDYGVLKNDEGIAYRGLFIIDGKGVLRQITVNDLPVGRSVDEALRLVQAFQYTDEHGEVCPAAWKPGRDTIKPNVDDSKEYFSKHN

SEQ ID NO: 53 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFNHLAWINTPRKEGGLGPLNIPLLGDVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 54 - protein, 298 aa, Homo sapiens
MAARRALHFVFKVGNRFQTARFYRDVLGMKVLRHEEFEEGCKAACNGPYDGKWSKTMVGFGPEDDHFVAELTYNYGVGDYKLGNDFMGITLASSQAVSNARKLEWPLTEVAEGVFETEAPGGYKFYLQNRSLPQSDPVLKVTLAVSDLQKSLNYWCNLLGMKIYEKDEEKQRALLGYADNQCKLELQGVKGGVDHAAAFGRIAFSCPQKELPDLEDLMKRENQKILTPLVSLDTPGKATVQVVILADPDGHEICFVGDEAFRELSKIDPEGSKLLDDAMAADKSDEWFAKHNKPKASG

SEQ ID NO: 55 - protein, 298 aa, Homo sapiens
MAARRALHFVFKVGNRFQTARFYRDVLGMKVLRHEEFEEGCKAACNGPYDGKWSKTMVGFGPEDDHFVAELTYNYGVGDYKLGNDFMGITLASSQAVSNARKLEWPLTEVAEGVFETEAPGGYKFYLQNRSLPQSDPVLKVTLAVSDLQKSLNYWCNLLGMKIYEKDEEKQRALLGYADNQCKLELQGVKGGVDHAAAFGRIAFSCPQKELPDLEDLMKRENQKILTPLVSLDTPGKATVQVVILADPDGHEICFVGDEAFRELSKMDPEGSKLLDDAMSADKSDEWFAKHNKPKASG

SEQ ID NO: 56 - protein, 504 aa, Homo sapiens
MRLTPFSLSTGNSFRYSRRLKKNIFGTAPALRVSEMSLRPSSRIFPCFSRNGLDFTIVITLAQPPVPGISFIVAKPRLFPGAGSAGCGLLERLFLSLLLGTGLRWCLRGCFPGARFCSTTSPEGHTTFTGLRRSARTQRLAQGPKPGPPAATVARQTSRVSPAPPCSLRPGLRHESAPSGIGDVTARGALRGLGCTVRVTAACGGNHGCSQMLHFVFKVGNRFQTARFYRDVLGMKVLRHEEFEEGCKAACNGPYDGKWSKTMVGFGPEDDHFVAELTYNYGVGDYKLGNDFMGITLASSQAVSNARKLEWPLTEVAEGVFETEAPGGYKFYLQNRSLPQSDPVLKVTLAVSDLQKSLNYWCNLLGMKIYEKDEEKQRALLGYADNQCKLELQGVKGGVDHAAAFGRIAFSCPQKELPDLEDLMKRENQKILTPLVSLDTPGKATVQVVILADPDGHEICFVGDEAFRELSKMDPEGSKLLDDAMAADKSDEWFAKHNKPKASG

SEQ ID NO: 57 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 58 - protein, 782 aa, Homo sapiens; identical to SEQ ID NO: 57

SEQ ID NO: 59 - protein, 426 aa, Homo sapiens
FNNHGLQVQQQQQRNLSLHEYMSMELLQEAGVSVPKGYVAKSPDEAYAIAKKLGSKDVVIKAQVLAGGRGKGTFESGLKGGVKIVFSPEEAKAVSSQMIGKKLFTKQTGEKGRICNQVLVCERKYPRREYYFAITMERSFQGPVLIGSSHGGVNIEDVAAETPEAIIKEPIDIEEGIKKEQALQLAQKMGFPPNIVESAAENMVKLYSLFLKYDATMIEINPMVEDSDGAVLCMDAKINFDSNSAYRQKKIFDLQDWTQEDERDKDAAKANLNYIGLDGNIGCLVNGAGLAMATMDIIKLHGGTPANFLDVGGGATVHQVTEAFKLITSDKKVLAILVNIFGGIMRCDVIAQGIVMAVKDLEIKIPVVVRLQGTRVDDAKALIADSGLKILACDDLDEAARMVVKLSEIVTLAKQAHVDVKFQLPI

SEQ ID NO: 60 - protein, 295 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGVHLISNVYGRSTSLKVVL

SEQ ID NO: 61 - protein, 335 aa, Homo sapiens
MKLPARVFFTLGSRLPCGLAPRRFFSYGTKILYQNTEALQSKFFSPLQKAMLPPNSFQGKVAFITGGGTGLGKGMTTLLSSLGAQCVIASRKMDVLKATAEQISSQTGNKVHAIQCDVRDPDMVQNTVSELIKVAGHPNIVINNAAGNFISPTERLSPNAWKTITDIVLNGTAFVTLEIGKQLIKAQKGAAFLSITTIYAETGSGFVVPSASAKAGVEAMSKSLAAEWGKYGMRFNVIQPGPIKTKGAFSRLDPTGTFEKEMIGRIPCGRLGTVEELANLAAFLCSDYASWINGAVIKFDGGEEVLISGEFNDLRKVTKEQWDTIEELIRKTKGS

SEQ ID NO: 62 - protein, 338 aa, Homo sapiens [sequence omitted]

SEQ ID NO: 63 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 64 - protein, 338 aa, Homo sapiens
MLSALARPASAALRRSFSTSAQNNAKVAVLGASGGIGQPLSLLLKNSPLVSRLTLYDIAHTPGVAADLSHIETKAAVKGYLGPEQLPDCLKGCDVVVIPAGVPRKPGMTRDDLFNTNATIVATLTAACAQHCPEAMICVIANPVNSTIPITAEVFKKHGVYNPNKIFGVTTLDIVRANTFVAELKGLDPARVNVPVIGGHAGKTIIPLISQCTPKVDFPQDQLTALTGRIQEAGTEVVKAKAGAGSATLSMAYAGARFVFSLVDAMNGKEGVVECSFVKSQETECTYFSTPLLLGKKGIEKNLGIGKVSSFEEKMISDAIPELKASIKKGEDFVKTLK

SEQ ID NO: 65 - protein, 224 aa, Homo sapiens
MDCREMDLYEDYQSPFDFDAGVNKSYLYLSPSGNSSPPGSPTLQKFGLLRTDPVPEEGEDVAATISATETLSEEEQEELRRELAKVEEEIQTLSQVLAAKEKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSAANASATTTEPLPEKTQESL

SEQ ID NO: 66 - protein, 276 aa, Homo sapiens [sequence omitted]

SEQ ID NO: 67 - protein, 184 aa, Homo sapiens [sequence omitted]

SEQ ID NO: 68 - protein, 184 aa, Homo sapiens
MDRGEQGLLRTDPVPEEGEDVAATISATETLSEEEQEELRRELAKVEEEIQTLSQVLAAKEKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSAANASATTTEPLPEKTQESL

SEQ ID NO: 69 - protein, 222 aa, Homo sapiens
RESPAEARRSSARRGGRSEPGRAAGGGAAEDTRRRAGDMDRGEQGLLRTDPVPEEGEDVAATISATETLSEKEQEELRRELAKVEEEIQTLSQVLAAKEKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSAANASATTTEPLPEKTQESL

SEQ ID NO: 70 - protein, 207 aa, Homo sapiens
MDRGEQGLLRTDPVPEEGEDVAATISATETLSEEEQEELRRELAKVEEEIQTLSQVLAAKEKHLAEIKRKLGINSLQELKQNIAKGWQDVTATSAYKKTSETLSQAGQKASAAFSSVGSVITKKLEDVKLQAFSHSFSIRSIQHSISMPAMRNSPTFKSFEEKVENLKSKVGGTKPAGGDFGEVLNSAANASATTTEPLPEKTQESL

SEQ ID NO: 71 - protein, 249 aa, Homo sapiens
MAPSRKFFVGGNWKMNGRKQSLGELIGTLNAAKVPADTEVVCAPPTAYIDFARQKLDPKIAVAAQNCYKVTNGAFTGEISPGMIKDCGATWVVLGHSERRHVFGESDELIGQKVAHALAEGLGVIACIGEKLDEREAGITEKVVFEQTKVIADNVKDWSKVVLAYEPVWAIGTGKTATPQQAQEVHEKLRGWLKSNVSDAVAQSTRIIYGGSVTGATCKELASQPDVDGFLVGGASLKPEFVDIINAKQ

(19) United States
(12) Patent Application Publication
(10) Pub. No.: US 2011/0294136 A1
(43) Pub. Date: Dec. 1, 2011

(54) BIOMARKER FOR DIAGNOSING PANCREATIC CANCER

(76) Inventors: Helmut Meyer, Recklinghausen (DE); Wolff Schmiegel, Bochum (DE); Barbara Sitek, Bochum (DE); Kai Stühler, Bochum (DE); Bence Sipos, Kiel (DE); Günter Klöppel, Kiel (DE); Ibrahim Alkatout, Kiel (DE); Stephan Hahn, Bochum (DE)

(21) Appl. No.: 12/312,954

(22) PCT Filed: Dec. 3, 2007
(86) PCT No.: PCT/DE2007/002174

§ 371 Date: Jan. 15, 2010

(30) Foreign Application Priority Data
Dec. 1, 2006 (DE) 10-2006-056-784.6

(51) Int. Cl. G01N 33/53; C40B 60/12

(52) U.S. Cl. 435/7.1; 506/39

(57) ABSTRACT

The invention relates to a method for diagnosing pancreatic cancer (PaCa) or the precursor diseases and/or concomitant diseases thereof, in particular pancreatic ductal adenocarcinoma (PDAC), pancreatic intraepithelial neoplasia (PanIN), pancreatic lesions, chronic pancreatitis (CP), including endocrine pancreatic tumors. In said method, the diagnosis is performed using selected biomarkers. The invention further relates to biomarker combinations suitable for carrying out said method, particularly for in vitro diagnosis.

The invention relates to a method for the diagnosis of pancreatic cancer (synonymous term: pancreatic carcinoma) (PaCa) and the precursor and/or concomitant illnesses thereof, particularly PDAC (pancreatic ductal adenocarcinoma), PanIN (pancreatic intraepithelial neoplasias), pancreatic lesions, CP (chronic pancreatitis), including endocrine tumors of the pancreas, where a determination is carried out using selected biomarkers. Furthermore, the invention relates to suitable combinations of biomarkers, particularly for in vitro diagnostics.

The 5-year-survival rate for pancreatic carcinoma of approx. 1% is the lowest of all cancer types (Parkin, D. M., F. Bray, et al. (2001). “Estimating the world cancer burden: Globocan 2000.” Int J Cancer 94(2): 153-6). Early diagnosis might increase the 5-year survival rate to 40% (Yeo, C. J. and J. L. Cameron (1998). “Prognostic factors in ductal pancreatic cancer.” Langenbecks Arch Surg 383(2): 129-33). Therefore, for diagnosis, the precursor diseases of pancreatic cancer need to be considered as well, such as PDAC (pancreatic ductal adenocarcinoma), PanIN (pancreatic intraepithelial neoplasias), pancreatic lesions, CP (chronic pancreatitis), including endocrine tumors of the pancreas. Especially PanID are associated with pancreatic lesions and differentiate them morphologically into PanIn 1A, 1B, 2, and 3 (Kern, S., R. Hruban, et al. (2001). “A white paper: the product of a pancreas cancer think tank.” Cancer Res 61(12): 4923-32). Pancreatic lesions have also been described for CP. Endocrine (benign or malignant) tumors of the pancreas, particularly neuroendocrine tumors, are relevant as well.

For the purpose of a useful therapy of pancreatic cancer or of precursor and/or concomitant illnesses thereof, particularly PDAC (pancreatic ductal adenocarcinoma), PanIN (pancreatic intraepithelial neoplasias), pancreatic lesions, CP (chronic pancreatitis), including endocrine tumors of the pancreas, there is a requirement of early diagnosis and differentiation in connection with the need for clinical decisions.

However, a drawback of the current diagnostic methods using the presently known markers is that the early and comprehensive identification of risk patients is unsuccessful, which is why diagnosis is incomplete or even too late.

An underlying objective of the invention is therefore to develop a method for diagnosis of pancreatic cancer or of precursor and/or concomitant illnesses thereof, enabling an improved early diagnosis and identification of risk patients as well as an improvement of the therapeutic success.

Another disadvantage is that often no sufficient sensitivity and/or specificity of the markers can be obtained in the art. For example, the early diagnosis of PDAC is associated with the significant problem of not having a specific biomarker. The most commonly used serum biomarker for pancreatic cancer is C-19-9, with a specificity of only 69-90%, since this marker can be detected in the blood in other diseases as well, particularly in chronic pancreatitis (Banfi et al. (1996) CA 19.9, CA 242 and CEA in the diagnosis and follow-up of pancreatic cancer, Int J Biol Markers, 77-81, Banfi et al (1993) Behavior of tumor markers CA19.9, CA195, CAM43, CA242, and TPS in the diagnosis and follow-up of pancreatic cancer, Clin Chem, 420-3).

The object is attained through a method for diagnosis of pancreatic cancer or precursor and/or concomitant illnesses thereof, whereby a determination of at least one polypeptide/proteins selected from the group

a.) Keratin 8 protein (SEQ ID No. 1), Vimentin (SEQ ID No. 2), Mitochondrial malate dehydrogenase (SEQ ID No. 3), Beta tropomyosin (SEQ ID No. 4), ACTG1 protein (SEQ ID No. 5), Thioredoxin delta 3 (SEQ ID No. 6), B Chain B Triosephosphate Isomerase (SEQ ID No. 7), Annexin A2 (SEQ ID No. 8), TPM4-ALK fusion oncoprotein type 2 (SEQ ID No. 9), Peptidylprolyl isomerase A (SEQ ID No. 10), Smooth muscle mysoin light chain (SEQ ID No. 11), Desmin (SEQ ID No. 12), Major vault protein 1 (SEQ ID No. 13), Heterogeneous nuclear ribonucleoprotein A1 (SEQ ID No. 14), S100A10 (SEQ ID No. 15), EF1a-like protein (SEQ ID No. 16), Regulatory myosin light chain long version (SEQ ID No. 17), Tropomyosin 1 alpha chain isoform 3 (SEQ ID No. 18), Tropomyosin 2 (beta) isoform 2 (SEQ ID No. 19), Myosin regulatory light chain MRCL3 (SEQ ID No. 20), Alpha-2-globin (SEQ ID No. 21), Tropomyosin 4 (SEQ ID No. 22), Transgelin (SEQ ID No. 23), Keratin 7 (SEQ ID No. 24), ACTB protein (SEQ ID No. 25), M2-type pyruvate kinase (SEQ ID No. 26), Actin related protein ⅔ complex subunit 5 (SEQ ID No. 27), Anterior gradient 2 homolog (AGR 2) (SEQ ID No. 28), Stratifin (14-3-3 sigma) (SEQ ID No. 29), Coactosin-like 1 (SEQ ID No. 30), Chaperonin heat shock 60 kD protein 1 (SEQ ID No. 31), Transgelin 2 (SEQ ID No. 32), Aldehyde dehydrogenase 1 (SEQ ID No. 33), Sarcomeric tropomyosin kappa (SEQ ID No. 34), Annexin A3 (SEQ ID No. 35), Delta-globin (SEQ ID No. 36), Serum albumin (SEQ ID No. 37), Protein PP4-X (Annexin A4) (SEQ ID No. 38), Crystallin (SEQ ID No. 39), Myosin regulatory light chain MRCL3 (SEQ ID No. 40)
or
group b.) aldehyde dehydrogenase 1 (SEQ ID No. 41), Aldehyde dehydrogenase 1A1 (SEQ ID No. 42), T-complex protein 1 subunit beta (SEQ ID No. 43), Apolipoprotein A4 (SEQ ID No. 44), Malate dehydrogenase mitochondrial precursor (SEQ ID No. 45), Voltage-dependent anion selective channel protein 1 (SEQ ID No. 46), glyceraldehydes-3-phosphate dehydrogenase (SEQ ID No. 47), uracil DNA glycosylase (SEQ ID No. 48), aging-associated-associated 9 protein (SEQ ID No. 49), Nipsnap homolog 3A (SEQ ID No. 50), peroxiredoxin 2 isoform b (SEQ ID No. 51), thiol-specific antioxidant protein (SEQ ID No. 52), enhancer protein (SEQ ID No. 53), Chromosome 17 open reading frame 25 (SEQ ID No. 54), hypothetical protein LOC51031 (SEQ ID No. 55), CGI-150 protein (SEQ ID No. 56), Gelsolin isoform a (SEQ ID No. 57), Gelsolin precursor (SEQ ID No. 58), ATP-specific succinyl-CoA synthetase beta subunit (SEQ ID No. 59), TAR DNA binding protein (SEQ ID No. 60), 2,4-dienoyl-CoA reductase mitochondrial precursor (SEQ ID No. 61), MDH2 (SEQ ID No. 62), heat shock protein beta-1 (SEQ ID No. 63), mitochondrial malate dehydrogenase precursor MDH-2 (SEQ ID No. 64), prostate and colon associated protein (SEQ ID No. 65), secretagogin (SEQ ID No. 66), TPD 52 (SEQ ID No. 67), tumor protein D52 (SEQ ID No. 68), N8 protein long isoform (Fragment) variant (SEQ ID No. 69), tumor protein D52 isoform 2 (SEQ ID No. 70), triosephosphate isomerase 1 (SEQ ID No. 71) or partial peptides or fragments thereof is carried out on a patient to be investigated (hereinafter referred to as method according to the invention”).

The proteins according to the invention are identified as potential biomarkers by means of a differential proteome analysis from ill pancreatic ductal tissue—five progression phases—in comparison to normal (healthy) pancreatic ductal tissue. Hereto, appropriate tissue samples were taken from ill patients. The samples were homogenized with lysis buffer in a hand-held homogenizer and removed from DNA and other cell material resulting in a protein concentrate.

The proteins were labeled with a dye and subject to a 2D gel electrophoresis with an isoelectric focusing in the first dimension and a SDS gel electrophoresis in the second dimension. The differential illustration (ill/healthy) is presented in tables (1 to 3), examples and figures showing different characteristic expressions (up- and down-regulated and read out by using the spots).

Further examination was carried out by means of LC-ESI-MS(/MS) (Liquid-Chromatographie-Electrospray-Ionization-Mass Spectrometry). In a first instance the proteins were fragmented in specific peptide fragments by means of trypsin within the gel, afore the samples were separated. Those were each other separated by means of reversed-phase HPLC and examined with mass spectrometry in order to identify each protein. It should be understood that other methods of mass spectrometry are also suitable like MALDI-TOF-MS.

The proteins in accordance with the invention (biomarkers) are identified as follows:

TABLE 1
group a.) with respect to PanIN

| | | Fold change in regard to normal epithelium | | | | | | 2-DE | | NCBI | | Sequence |
|---|---|---|---|---|---|---|---|---|---|---|---|---|
| Spot | Up-regulated | PanIN | PanIN | PanIN | PanIN | | NCBI | | Mᵣ | | Mᵣ | coverage |
| No | Proteins | 1A | 1B | 2 | 3 | Carcinoma | accession | p/ | (kDa) | p/ | (kDa) | (%) |

Early up-/down-regulated spots

| | | | | | | | | | | |
|---|---|---|---|---|---|---|---|---|---|---|
| 1944 | Keratin 8 protein | 2.3 | | | gi\|33875698 | 4.8 | 39.8 | 5.5 | 55.8 | 9.1 |
| 1635 | Vimentin | | 2.3 | | gi\|7576229 | 4.7 | 46.0 | 5.1 | 53.7 | 13.3 |
| 1813 | Mitochondrial malate | | 2.7 | | gl\|12804929 | 7.6 | 42.8 | 9.1 | 35.5 | 12.7 |
| | dehydrogenase | | | | | | | | | |
| 2206 | Beta tropomyosin | | 2.1 | | gi\|6573280 | 4.8 | 34.4 | 4.7 | 29.9 | 28.0 |
| 1962 | ACTG1 protein | | 2.9 | | gi\|40226101 | 5.7 | 39.5 | 5.4 | 29.4 | 11.0 |
| 2925 | Thioredoxin delta 3 | 2.8 | 2.2 | 3.1 | gi\|3153859 | 5 | 16.8 | 5.7 | 9.3 | 26.2 |
| 2330 | B Chain B, Triosephosphate | | | | gi\|999893 | 6.5 | 32.7 | 7.7 | 38.6 | 9.1 |
| | Isomerase | | | | | | | | | |
| 2126 | Annexin A2 | | | 2.3 | gi\|16306978 | 5.7 | 36.0 | 5.5 | 29.8 | 19.1 |
| 2154 | TPM4-ALK fusion | −2.1 | | −3.4 | gi\|10441386 | 4.7 | 35.5 | 4.8 | 27.5 | 49.8 |
| | oncoprotein type 2 | | | | | | | | | |
| 2639 | Peptidylprolyl isomerase A | | −2.3 | | gi\|62205349 | 7.3 | 27.1 | 7.9 | 11.4 | 41.9 |
| 2765 | Smooth muscle mysoin light | | | −4.1 | gl\|189022 | 4.4 | 22.5 | 4.7 | 12.9 | 21.6 |
| | chain | | | | | | | | | |
| 821 | Vimentin | | | −2.2 | gi\|7576229 | 5.3 | 62.6 | 5.1 | 53.7 | 41.0 |

Late up-/down-regulated spots

| | | | | | | | | | | |
|---|---|---|---|---|---|---|---|---|---|---|
| 999 | Desmin | 2.4 | 3.0 | | gi\|1408188 | 5.3 | 58.3 | 5.2 | 53.5 | 24.2 |
| 1243 | Major vault protein (MVP) | | 5.1 | | gi\|15990478 | 5.9 | 53.6 | 5.3 | 99.3 | 3.1 |
| 1836 | Heterogeneous nuclear | | 3.0 | | gi\|14043070 | 8.1 | 42.6 | 9.2 | 38.7 | 9.1 |
| | ribonucleoprotein A1 | | | | | | | | | |
| 3022 | S100A10 | | 2.8 | | gi\|4388970 | 7.2 | 14.2 | 7.5 | 11.1 | 1.7 |
| 2697 | EF1a-like protein | | 21.0 | 14.1 | gi\|24210508 | 7.9 | 25.4 | 7.2 | 46.4 | 4.9 |
| 2711 | Regulatory myosin light | −1.9 | −2.1 | | gi\|33338062 | 4.7 | 24.9 | 4.6 | 19.9 | 17.4 |
| | chain long version | | | | | | | | | |
| 1926 | Tropomyosin 1 alpha chain | −2.1 | −2.6 | | gi\|63252896 | 4.7 | 40.3 | 4.6 | 32.7 | 22.5 |
| | isoform 3 | | | | | | | | | |
| 823 | Vimentin | −2.7 | −3.2 | | gi\|7576229 | 5.2 | 62.4 | 5.1 | 53.7 | 48.5 |
| 1738 | Tropomyosin 2 (beta) | −3.0 | −2.6 | | gi\|55859703 | 4.3 | 30.0 | 4.6 | 33.0 | 67.6 |
| | isoform 2 | | | | | | | | | |
| 2649 | Myosin regulatory light chain | | −1.8 | | gi\|62896697 | 4.8 | 26.6 | 4.5 | 19.8 | 17.5 |
| | MRCL3 | | | | | | | | | |
| 2946 | Alpha-2-globin | | −3.5 | | gi\|1335076 | 7.9 | 16.4 | 8.7 | 15.1 | 39.7 |
| 2085 | Tropomyosin 4 | | | −1.6 | gi\|12803959 | 4.6 | 36.7 | 4.7 | 28.6 | 40.3 |
| 2217 | A25074 vimentin | | | −6.9 | gi\|7576229 | 7.1 | 34.4 | 5.1 | 53.7 | 24.5 |
| 2547 | Transgelin | −2.7 | −1.8 | 3.2 | gi\|62205326 | 7.5 | 28.5 | 8.9 | 22.6 | 64.7 |

Constant up-/down-regulated spots

| | | | | | | | | | | | | |
|---|---|---|---|---|---|---|---|---|---|---|---|---|
| 738 | Keratin 7 | 1.7 | 1.9 | | | 4.0 | gi\|60655723 | 5.5 | 64.4 | 5.4 | 51.4 | 44.1 |
| 1347 | ACTB protein | | 3.1 | 4.2 | 3.2 | 2.4 | gi\|15277503 | 5.9 | 51.5 | 5.6 | 40.2 | 20.0 |
| 2921 | Thioredoxin delta 3 | 1.9 | 2.5 | 2.0 | 1.4 | | gi\|3153859 | 5.0 | 16.9 | 5.7 | 9.3 | 36.9 |
| 1276 | ACTB protein | | 3.9 | 1.8 | 4.8 | | gi\|15277503 | 5.9 | 52.6 | 5.6 | 40.2 | 17.8 |
| 1340 | M2-type pyruvate kinase | | 2.3 | | 3.1 | | gi\|33286422 | 6.0 | 51.5 | 8.7 | 58.0 | 8.7 |
| 2781 | Actin related protein 2/3 | | 2.0 | 1.9 | 2.1 | 1.9 | gi\|56204524 | 5.9 | 21.8 | 5.6 | 16.6 | 34.4 |
| | complex subunit 5 | | | | | | | | | | | |
| 2793 | Anterior gradient 2 homolog | 6.0 | 11.3 | 8.6 | | 3.8 | gi\|37183136 | 8.1 | 21.5 | 9.5 | 20.0 | 14.3 |
| | (AGR 2) | | | | | | | | | | | |
| 2799 | Anterior gradient 2 homolog | 3.3 . | 5.8 | 5.7 | 5.4 | 6.7 | gi\|37183136 | 8.1 | 21.1 | 9.5 | 20.0 | 4.0 |
| | (AGR 2) | | | | | | | | | | | |
| 2437 | Annexin A2 | 3.3 | 10.1 | 6.9 | 3.7 | 3.3 | gi\|16306978 | 5.5 | 30.5 | 7.7 | 38.6 | 11.2 |
| 2192 | Stratifin (14-3-3 sigma) | | 2.9 | 2.0 | 4.0 | | gi\|7981260 | 4.6 | 34.7 | 4.7 | 27.8 | 35.1 |
| 2843 | Coactosin-like 1 | | 2.4 | | 2.1 | 1.4 | gi\|27695621 | 5.5 | 19.3 | 5.4 | 16.0 | 31.0 |
| 734 | Chaperonin; heat shock | | 1.9 | | | 2.8 | gi\|6996447 | 5.4 | 64.5 | 5.7 | 61.1 | 22.2 |
| | 60 kD protein 1 | | | | | | | | | | | |
| 2608 | Transgelin 2 | 2.6 | | | 3.6 | | gi\|55960373 | 6.6 | 27.6 | 8.4 | 22.4 | 15.1 |
| 791 | Aldehyde dehydrogenase 1 | −2.2 | −3.3 | −2.7 | −3.0 | −5.1 | gi\|2183299 | 6.4 | 63.4 | 6.3 | 54.8 | 7.8 |
| 819 | Vimentin | −2.2 | −3.5 | −3.9 | −5.6 | −2.1 | gi\|7576229 | 5.1 | 62.5 | 5.1 | 53.7 | 34.5 |
| 820 | Vimentin | −1.9 | −2.1 | −3.2 | −4.9 | | gi\|7576229 | 5.2 | 62.5 | 5.1 | 53.7 | 41.0 |
| 1828 | Sarcomeric tropomyosin | −2.9 | −3.2 | | −3.0 | −2.4 | gi\|49660012 | 5.4 | 42.5 | 4.5 | 32.6 | 46.1 |
| | kappa; TPM1-kappa | | | | | | | | | | | |
| 1852 | Annexin A3 | | −1.7 | −2.7 | | −4.3 | gi\|12654115 | 5.8 | 42.0 | 5.6 | 36.4 | 12.7 |
| 2879 | Delta-globin | −3.1 | | | −8.2 | −3.1 | gi\|18462107 | 7.6 | 18.1 | 8.0 | 16.1 | 20.4 |
| 923 | Serum albumin | | −2.1 | −2.1 | −2.4 | | gi\|28592 | 5.7 | 60.2 | 6.1 | 69.4 | 7.1 |
| 1811 | Protein PP4-X (Annexin A4) | | −5.5 | −4.8 | | −7.6 | gi\|189617 | 5.9 | 42.8 | 5.6 | 36.1 | 29.3 |
| 2022 | Crystallin | | −8.0 | | −14.5 | −10.3 | gi\|28634 | 6.1 | 38.3 | 5.5 | 12.4 | 28.8 |
| 2660 | Myosin regulatory light chain | −1.8 | | −2.0 | −1.8 | | gi\|2605594 | 4.9 | 26.3 | 4.6 | 19.7 | 17.4 |
| | MRCL3 | | | | | | | | | | | |

NCBI: National Centre for Biotechnology Information

TABLE 2
Part of group b. ) with respect to up-regulated
proteins (Biomarkers) in malignant samples of pancreatic tumors

| | Spotnummer | T-test | Faktor | Protein |
|---|---|---|---|---|
| 1 | 895 | 0.06536 | −8.5 | aldehyde dehydrogenase 1A1 [Homo sapiens] |
| 2 | 986 | 0.04567 | −8.2 | aldehyde dehydrogenase 1A1 [Homo sapiens] |
| 3 | 988 | 0.01732 | −7.5 | T-complex protein 1 subunit beta |
| 4 | 1388 | 0.004622 | −2.4 | apolipoprotein A4 |
| 5 | 1523 | 0.0085 | −3.57 | Malate dehydrogenase, mitochondrial precursor |
| | | | | Voltage-dependent anion-selective channel protein 1 |
| 6 | 1539 | 0.04386 | −1.6 | glyceraldehyde-3-phosphate dehydrogenase |
| | | | | uracil DNA glycosylase [Homo sapiens] |
| | | | | aging-associated gene 9 protein [Homo sapiens] |
| 7 | 2166 | 0.02121 | −3.2 | Nipsnap homolog 3A [Homo sapiens] |
| 8 | 2314 | 0.03199 | −1.4 | peroxiredoxin 2 isoform b [Homo sapiens] |
| | | | | thiol-specific antioxidant protein [Homo sapiens] |
| | | | | enhancer protein |
| 9 | 2117 | 0.003302 | −2 | Chromosome 17 open reading frame 25 [Homo sapiens] |
| | | | | hypothetical protein LOC51031 [Homo sapiens] |
| | | | | CGI-150 protein [Homo sapiens] |
| | | | | Apolipoprotein A-IV |

TABLE 3
Part of group b.) with respect to up-regulated
Proteins (biomarkers) of benign samples of pancreatic tumors

| | Spotnummer | T-test | Faktor | Protein |
|---|---|---|---|---|
| 1 | 414 | 0.001686 | 3.9 | Gelsolin, isoform a [Homo sapiens] |
| 2 | 420 | 0.004877 | 2.3 | Gelsolin precursor |
| 3 | 1142 | 0.0141 | 3.9 | ATP-specific succinyl-CoA synthetase beta subunit |
| | | | | TAR DNA binding protein [Homo sapiens] |
| 4 | 1707 | 0.04 | 2.41 | 2,4-dienoyl-CoA reductase, mitochondrial precursor |
| 5 | 1708 | 0.03963 | 2.8 | MDH2 [Homo sapiens] |
| 6 | 1718 | 0.01763 | 3.1 | Heat-shock protein beta-1 |
| 7 | 1721 | 0.03081 | 3.2 | mitochondrial malate dehydrogenase precursor |
| | | | | MDH2 [Homo sapiens] |
| 8 | 1970 | 0.04907 | 3.9 | prostate and colon associated protein [Homo sapiens] |
| | | | | secretagogin [Homo sapiens] |
| | | | | TPD52 [Homo sapiens] |
| | | | | tumor protein D52 [Homo sapiens] |
| | | | | N8 protein long isoform (Fragment) variant |
| | | | | [Homo sapiens] |
| | | | | tumor protein D52 isoform 2 [Homo sapiens] |
| 9 | 2049 | 0.05658 | 2.5 | triosephosphate isomerase 1 [Homo sapiens] |

The invention refers also to such amino acid sequences of SEQ ID No. 1 to SEQ ID No. 71 (polypeptide, proteins), having a sequence identity or homology of 70% and more, preferably 80% and more, most preferably 90-95%.

Likewise are included such analogous amino acid sequences having although due to a replacement of one or more amino acid(s) the desired function of a biomarker for diagnosis of pancreatic cancer. Expressly included according to the invention are in particular partial peptides or fragments of SEQ ID No. 1 to SEQ ID No. 71.

In a further preferred embodiment of the invention combinations of biomarkers according to the invention are advantageously (Sub-combinations of the above entirety of all biomarkers according to the invention) for diagnosis. Particularly preferred are such combinations within the group

a.) comprising at least Stratifin (14-3-3 sigma) (SEQ ID No. 29) and/or Vimentin (SEQ ID No. 2) and/or Major vault protein 1 (SEQ ID No. 13) and/or Anterior gradient 2 homolog (AGR 2) (SEQ ID No. 28), and/or S100A10 (SEQ ID No. 15) and/or EF1a-like protein (SEQ ID No. 16) and/or Annexin A2 (SEQ ID No. 8) and/or Annexin A4 (SEQ ID No. 38).

The term “pancreatic cancer” in accordance with the invention encompasses also precursor and/or concomitant illnesses thereof, in particular PDAC (Pancreatic ductal adenocarcinoma), PanIN (pancreatic intraepithelial neoplasias), pancreatic lesions, CP (chronic pancreatitis), including endocrine pancreatic tumors, particularly pancreatic tumors und pancreatic neoplasm.

The invention therefore further relates to the identification of patients with increased risk and/or unfavorable prognosis of pancreatic cancer, particularly by symptomatic and/or asymptomatic patients.

The method according to the invention thus allows clinical decisions resulting in rapid therapeutic success and avoidance of mortalities. Such clinical decisions also include further treatment with medicaments for treatment or therapy of pancreas cancer. Clinical decisions of this type likewise include further treatment by means of pharmaceuticals for the treatment or therapy of pancreatic cancer.

Therefore, the invention relates also to a method for diagnosis of patients having pancreatic cancer for carrying out clinical decisions, like further treatment and therapy by means of medicaments.

In one further preferred embodiment of the method according to the invention, diagnosis is carried out for prognosis, differential diagnostic early detection and identification, severity assessment, and prognostic assessment in conjunction with therapy.

In one further preferred embodiment, the invention relates to a method for diagnostics for early or differential diagnosis or prognosis of pancreatic cancer or a precursor illness, wherein the biomarker is determined on a patient to be examined.

In one embodiment of the method according to the invention, tissue samples or bodily fluid (blood, plasma pancreatic secretion) is withdrawn from the patient to be examined, and the diagnosis is made in vitro/ex vivo, i.e. outside the human or animal body. As a result of the determination of the marker according to the invention high sensitivity and specificity for pancreatic cancer or precursor and/or concomitant illnesses thereof are achieved and diagnosis may be performed based on the quantity present or its shifting (level: increase/decrease) in at least one patient sample.

In a further embodiment of the invention, for an in vitro diagnosis the method according to the invention may be carried out by means of parallel or simultaneous determinations of the markers (for example, using multititer plates containing 96 or more cavities), wherein the determinations are carried out for at least one patient sample.

In a further embodiment, the method according to the invention may be carried out by means of 2D-elektrophoresis, wherein in a first dimension an isoelectric focusing and in the second dimension a SDS gel electrophoresis are conducted (This is understood in the broadest sense as proteome research (“proteomics”)).

In a further embodiment, the method according to the invention and determinations therefor may be carried out using a rapid test (for example, a lateral flow test) in either single- or multi-parameter determinations.

In a further embodiment, the method according to the invention may be carried out in-vivo, wherein the biomarkers are detected with a probe, particularly with an antibody, having a marked contrast agent and which are detectable with an image making suitable detector (“Molecular Imaging”) (Ralph Weissleder, Molecular Imaging in Cancer, Science, Vol. 312, 1168 (2006)).

The invention further relates to the use of the biomarker according to the invention for diagnosis and/or prognosis and/or for early or differential diagnosis of myocardial infarction of pancreatic cancer or precursor and/or concomitant illness thereof.

A further object is to provide a corresponding diagnostic device for carrying out the methods according to the invention.

Within the scope of the invention, such a diagnostic device, in particular an array or assay (for example, immunoassay, ELISA, etc.), is understood in the broadest sense as a device for carrying out the methods according to the invention, particularly a protein biochip (U.S. Pat. No. 6,346,413B1. US20050014292). The invention further relates to a kit for carrying out the methods according to the invention, particularly containing detection reagents and further adjuvants. Such detection reagents include antibodies, for example.

The detection and the quantification of the biomarkers according to the invention may also be performed with the aid of further protein diagnostic methods known to those skilled in the art, in particular employing radioactive or fluorescence-marked antibodies. In particular, bioanalytical methods suitable for this purpose are to be cited here, such as immunohistochemistry, antibody arrays, luminex, ELISA, immunofluorescence, and radio immunoassays as well as further bioanalytical methods suitable for this purpose, such as mass-spectrometry methods, e.g., MRM (multi-reaction monitoring) or AQUA (absolute quantification), with the aid of which the biomarkers may be quantitatively measured.

The following examples and figures are used for a more detailed explanation of the invention, but do not limit the invention to said examples and figures.

### EXAMPLES AND FIGURES

### Microdissection

The tissue samples were obtained from surgical patients of the General Surgery Department of the University Hospital Schleswig-Holstein, Campus Kiel (German). Tumor tissues from ductal pancreatic cancer and peritumoral parenchyma were shock frozen at −80° C. immediately postsurgically and stored thereafter. For visualization of normal pancreatic ducts and PanINs, 5 μm thick frozen sections were prepared of the peritumoral pancreas parenchyma, briefly fixed in ethanol (Merck, Darmstadt, Germany), stained with hematoxylin-eosin and subsequently evaluated by a pathologist. The PanINs were classified according to accepted criteria (Hruban, R. H., N. V. Adsay, et al. (2001). “Pancreatic intraepithelial neoplasia: a new nomenclature and classification system for pancreatic duct lesions.” Am J Surg Pathol 25(5): 579-86). Serial tissue block sections (10 μm) containing the required PanIN lesions were obtained. For the 2-D electrophoresis, the tissue sections were stained only with hematoxylin and immediately stored at −20° C. The PanIN lesions were microdissected under a microscope (BH2, Olympus, Wetzlar, Germany) using a sterile injection needle (size 0.65×25 mm, Braun company, Melsungen, Germany). Primarily medium sized interlobular ducts were selected, in order to avoid contamination with periductal mesenchymal and acinar tissue. The microdissected cells were taken up in 100 μL lysis buffer (Tris-Cl 30 mM; thiourea 2M; urea 7M; CHAPS 4%, pH 8.0) and treated on ice in an ultrasonic bath immediately after microdissection (6×10 s pulses; ultrasonic cleaner, VWR Darmstadt, Darmstadt).

Preparation of the Reference Proteome

For generation of the reference proteome, 100 mg adenocarcinoma tissue was homogenized in 148 μL lysis buffer (Tris-Cl 30 mM; thiourea 2M; urea 7M; CHAPS 4%, pH 8). Then the samples were sonicated (6×10 pulses, on ice) and centrifuged (12.000×g for 5 min). Protein determination was performed using a protein assay (Bio-Rad).

### Protein Labeling

The samples, each with 1000 microdissected cells in 100 μL lysis buffer, were reduced by addition of 2 nmoles TCEP, and were then incubated at 37° C. for 1 h in the dark. The saturation dyes Cy3 and Cy5 were first diluted with DMF (2 nmol/μL; Sigma) and were then added to the reduced samples in a concentration of 4 nmoles. The incubation took place at 37° C. for 30 min in the dark. To stop the labeling reaction, 10 μL DTT (1.08 g/mL; Bio-Rad) was added. Then, 10 μL Ampholine 2-4 (GE Healthcare) was added to each sample.

### Two-Dimensional Gel Electrophoresis

For separation of the proteins in the first dimension, carrier ampholyte-based IEF (slab gels 20 cm×1.5 mm) was conducted according to Klose and Kobalz (Klose, J. and U. Kobalz (1995). “Two-dimensional electrophoresis of proteins: an updated protocol and implications for a functional analysis of the genome.” Electrophoresis 16(6): 1034-59). After completion of a voltage program with 21.25 hrs, the ejected cylindrical gels were incubated in equilibration buffer (125 mM Tris, 40% (w/v) glycerin, 3% (w/v) SDS, 65 mM DTT, pH 6.8) for 10 min. The second dimension was obtained in an Desaphor VA 300 system with polyacrylamide gels (15.2% acrylamide (total), 1.3% bisacrylamide) (Klose and Kobalz 1995 (supra)). The cylindrical gels were applied to the polyacrylamide gels (20 cm×30 cm×1.5 mm) and fixed with 1% agarose containing 0.01% (w/v) bromophenole blue dye (Riedel deHaen, Seelze, Deutschland). The gel system used for protein identification (IEF: 20 cm×1.5 mm, SDS-PAGE: 20 cm×30 cm×1.5 mm) was processed under equal conditions. For this procedure, the MS-compatible silver staining protocol according to Nesterenko et al. was used (Nesterenko, M. V., M. Tilley, et al. (1994). “A simple modification of Blum's silver stain method allows for minutes detection of proteins in polyacrylamide gels.” J Biochem Biophys Methods 28(3): 239-42).

### Image Acquisition and Analysis

For image acquisition with the Typhoon 9400 fluorescence scanner (Amersham Biosciences/GE Healthcare) the gels remained between the glass plates. The excitation wave length and the emission filters were selected specifically for the respective fluorescence dyes according to the manual. Prior to the image analysis with the DeCyder software (Amersham Biosciences/GE Healthcare) the images were cropped using the ImageQuant™ software (Amersham Biosciences/GE Healthcare). Intra-gel spot detection and quantification took place using the Differential In-gel Analysis (DIA) mode of the DeCyder software. The estimated spot number was set to 3000. As an exclusion filter, an increase of the spot slope of more than 1.6 was selected. For determination of the reference proteome the matching rates between microdissected PDAC cells, a pancreatic cell lines pool, and PDAC tumor tissue were determined for various gel areas.

### In-Gel Digest and Protein Identification Using NanoLC-ESI-MS/MS

The spots were punched out manually from a preparative gel. In order to determine the position of the spots in the gel, a true to scale gel print was placed underneath the gel after image acquisition. Then, the spots were digested in the gel with trypsin (Promega, Mannheim, Germany), and the peptides were extracted as described in Schaefer et al. (Schaefer, H., J. P. Chervet, et al. (2004). “A peptide preconcentration approach for nano-high-performance liquid chromatography to diminish memory effects.” Proteomics 4(9): 2541-4; Schaefer, H., K. Marcus, et al. (2003). “Identification of phosphorylation and acetylation sites in alphaA-crystallin of the eye lens (mus musculus) after two-dimensional gel electrophoresis.” Anal Bioanal Chem 376(7): 966-72). For peptide analytics, a system consisting of FAMOS™ (automatic sampler), Switchos™ (loading pump and switch valves), and Ultimate™ (separation pump and UV detector) (LC Packings Dionex, Amsterdam, Niederlande), coupled on-line with an ion-trap mass spectrometer LCQ Deca XP (Thermo Electron, San Jose, Calif., USA) and equipped with a nanoelectrospray ion source (PicoView™100, New Objective Inc., Woburn, Mass., USA), and SilicaTips™ (FS360-20-10-D, New Objective Inc.) were used.

For protein identification, the MS/MS spectra were searched against the NCBI protein sequence sub-database (human) (http://www.ncbi.nlm.nih.gov) using the SEQUEST™ algorithm and accounting for the following search parameters: mass tolerance ±1.5 Da for parent and fragment ions. Cy3 modification of all cysteins. One overread trypsin cutting site. Proteins with a SequestMetaScore (Proteinscape™) larger than 10 with 3 or more peptides were considered as identified.

### Preparation of Tissue Arrays

For normal pancreatic ducts as well as for PanINs, one 1.5 mm thick tissue cylinder (two for ductal adenocarcinomas) was punched out of each representative area and embedded in paraffin reception blocks, so 300 cylinders with pancreatic tissues (in altogether 6 tissue arrays) as well as two control cylinders each with healthy tonsil tissue were processed in total. Processing took place using an MTA1 tissue arrayer instrument (Beecher Instruments, Sun Prairie, Wis., USA). Normal pancreatic ducts and the PanIN ducts were derived from 12 pancreases of healthy suicide victims that had been autopsied at the Pathology Department of the Semmelweis University in Budapest, Hungary (approval number: 140-1/1996), and from 81 pancreases that had been removed by surgical resection of gastrointestinal and pancreatic tumors in surgical departments at the university hospitals in Kiel and Dresden, Germany. For the tissue arrays of the pancreatic cancer, tissue blocks of 48 pancreases were used that had been removed in the surgical university clinic, Kiel, Germany.

### Immunohistochemistry

All investigations were conducted on formalin-fixed paraffin-embedded tissue. 3 μm thin sections were deparaffinized and rehydrated. Then, immunohistochemical stainings were performed according to the established method. Prior to the application of the primary antibody a 20 min. serum block was performed. The murine anti-14-3-3-sigma antibody (Acris, 1.N.6., 2.5 μg/μL, 1:40), the anti-LRP/MVP antibody (Kamiya Biomedical Company, 1032, 0.5 μg/μL, 1:400) and the rabbit anti-AGR2 antibody (Imgenex, 10 μg/μL, 1:50) were used as primary antibodies. The development of the signal was conducted using a mouse or rabbit staining kit (Vectastain Elite Peroxidase kit, PK-6102, Vector Laboratories, Burmingame, USA). As a negative control, the primary antibody was omitted.

### Evaluation of the Immunohistochemical Stainings

The intensity of the staining was classified into mild, moderate und strong (with a score of 1, 2, or 3, respectively). The stained areas were estimated in percent in terms of pancreatic ducts or tumor regions, and also classified into scores (<10%=1, 10-50%=2, 51-80%03, >80%=4). The final score was determined from the product of the staining intensity and the percentage of positively stained cells (minimum 0, maximum 12) (Remmele, Hildebrand et al. 1986).

### Statistics

Average values of the immunohistochemically determined scores of the normal pancreatic ducts, the various PanIN lesions as well as the ductal adenocarcinoma were compared using the Mann-Whitney U and Kruskal-Wallis H tests. A level of significance of 0.05 was applied to all statistical tests that were conducted. For multiple comparisons, the p-value was modified according to Bonferroni. All statistical calculations were performed using the SPSS 10.1 software. For identification of the biomarker candidates for pancreatic tumor progression, a differential proteome analysis of microdissected cells from PanIN lesions, PDAC and normal pancreatic ducts was performed. For this approach, tumors from 9 pancreas cancer patients, each providing 4-9 samples per lesion, were examined. The identified differential biomarkers were immunohistochemically validated with samples (tissue arrays) from 130 patients.

### Expression Profiles of the Differential Proteins

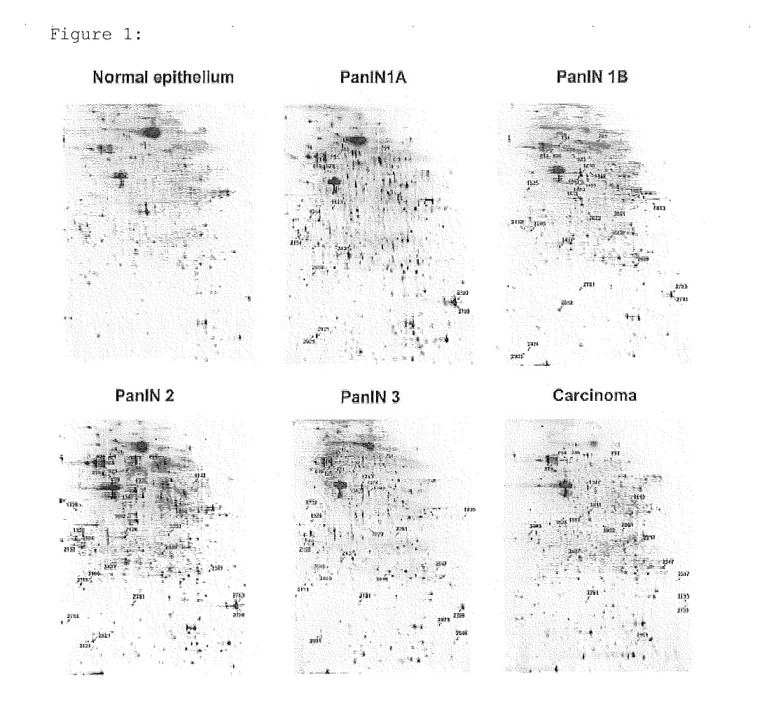

In the differential proteome analysis via 2-D electrophoresis, 86 different protein spots showing differential expression were detected in total. Among these, 19 spots in the PanIN 1A lesion, 37 in the PanIN 1B lesion, 40 in the PanIN 2 lesion, 39 in the PanIN 3 lesion, and 32 in PDAC were regulated differentially compared to normal pancreatic ducts (p<0.05, regulation factor >1.6). FIG. 1 shows one representative gel for each tumor stage, including the regulated protein spots.

For identification of the differential protein spots, the reference proteome of the pancreatic tumor tissue was used, the proteome pattern of which being highly consistent with the proteome of the microdissected material (>91%). Using LC-ESI-MS/MS, 38 non-redundant proteins in total could be identified (Table 1).

### Immunohistochemical Validation of Proteome Data

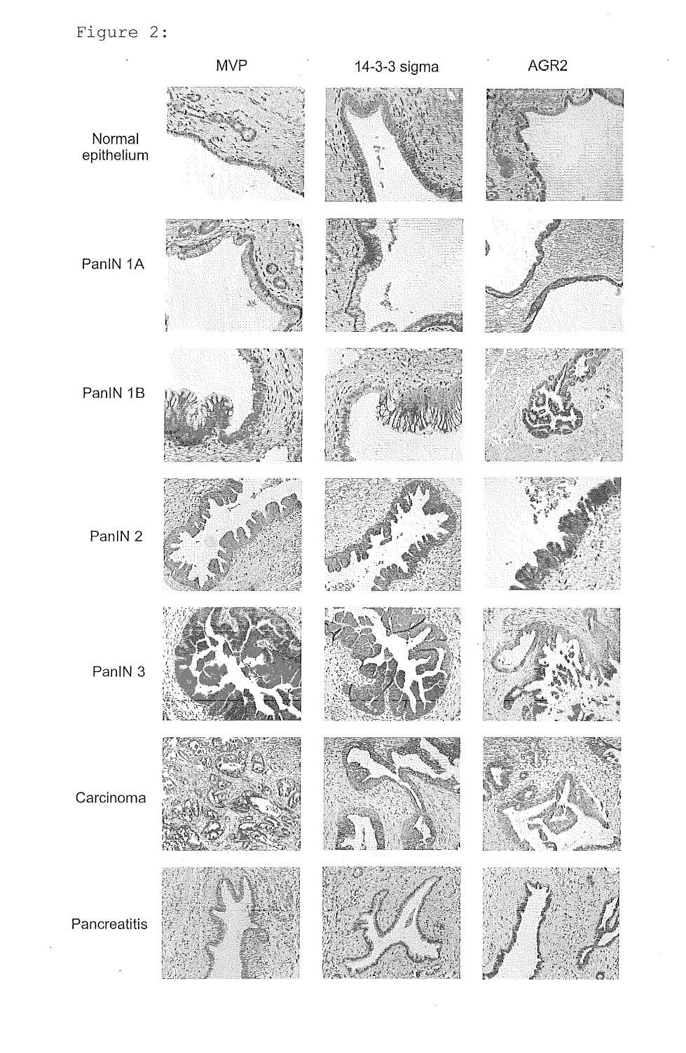

In order to be able to select proteins for immunohistochemical validation, their respective expression profiles during tumor progression were considered. Therefore, the differential protein spots were divided into 3 groups: 1) protein spots showing early regulation in the PanIN 1A and PanIN 1B lesions; 2) consistently modified protein spots throughout tumor progression; 3) protein spots with differential expression in an advanced tumor stage (PanIN 2 to PDAC) (see Table 1). Furthermore, the potential role of the proteins in tumor biology was taken as another criterion for immunohistochemical validation. Initially, among the 38 non-redundant proteins, seven were selected for validation in 130 patients: AGR2, MVP, stratifin, annexin A2, EFla-like protein, annexin A4 and S100A10. The proteome data could be confirmed for six of these proteins. The comparison of the proteome data and the validation is illustrated for three proteins: 14-3-3 sigma, MVP, and AGR2 (FIGS. 2, 3, and 4).

### Immunohistochemical Expression Profile of MVP

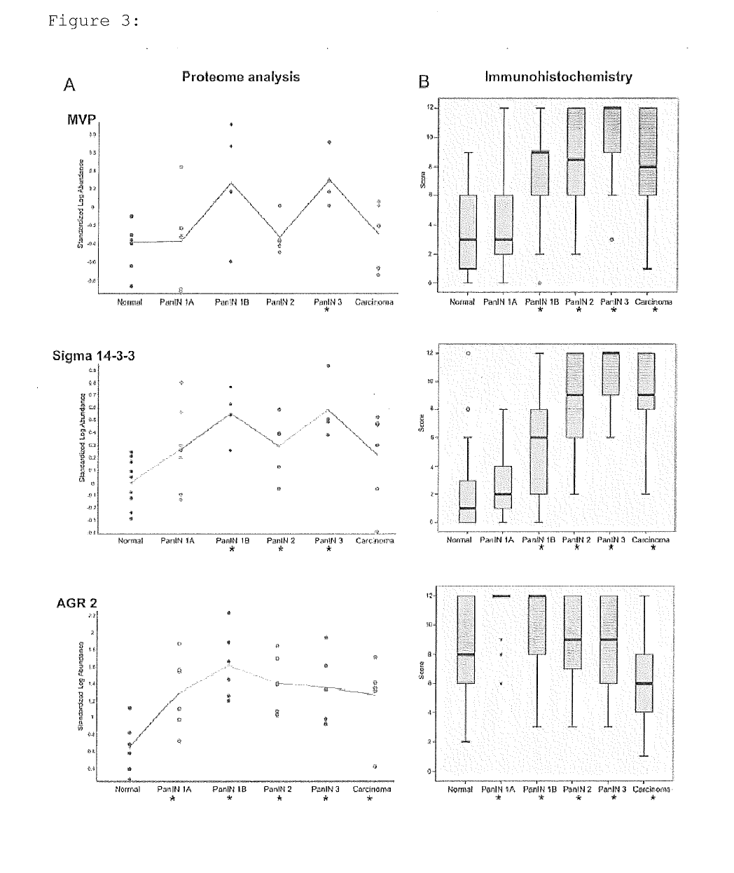

The MVP antibody stainings revealed an intra-cytoplasmic staining reaction. The average scores for MVP staining were as follows: normal ducts 3.70 (standard deviation 3.0, range 0-9); PanIN-1a 4.60 (standard deviation 3.2, range 0-12); PanIN-1b 7.82 (standard deviation 3.2, range 0-12); PanIN-2 7.93 (standard deviation 3.8, range 2-12); PanIN-3 10.00 (standard deviation 2.8, range 3-12) as well as ductal adenocarcinomas 8.32 (standard deviation 3.0, range 1-12) (FIG. 2). The scores of the various disease groups were significantly different (Kruskal-Wallis test, p<0.001). PanIN-1B, PanIN-2, PanIN-3, and PDAC showed a significantly higher MVP expression than normal pancreatic ducts (Mann-Whitney U test, p<0.001). Between PanIN-1B, PanIN-2, PanIN-3, and PDAC, no statistically significant differences could be detected (Kruskal-Wallis test, p=0.110). Increased MVP expression in PanIN-3 was detected by proteome analysis, as well as immunohistochemically (FIGS. 3 A, B).

### Immunohistochemical Expression Profile of 14-3-3 Sigma

Staining of the tissue arrays with the 14-3-3-sigma antibody displayed a primarily intra-cytoplasmic and less membrane-based staining reaction. The average scores for the 14-3-3 sigma staining were as follows: normal pancreatic ducts 2.04 (standard deviation 3.1, range 0-12); PanIN-1A 2.80 (standard deviation 2.6, range 0-8); PanIN-1B 5.30 (standard deviation 3.8, range 0-12); PanIN-2 8.34 (standard deviation 3.1, range 2-12); PanIN-3 10.61 (standard deviation 1.9, range 6-12), and PDAC 9.61 (standard deviation 2.8, range 2-12) (FIG. 2). Expression of 14-3-3-sigma was significantly different comparing the various groups (Kruskal-Wallis test, p<0.001). The expression of the 14-3-3-sigma protein was significantly increased in PanIN-1B compared to normal ducts and PanIN-1A (Mann-Whitney U test, p<0.001). Furthermore, the 14-3-3-sigma protein was expressed significantly stronger in PanIN-2, PanIN-3 and PDAC compared to PanIN-1B (Mann-Whitney U test, p<0.001). The results of the proteome analysis and the results of the immunohistochemical evaluation revealed a similar 14-3-3-sigma expression profile for PanIN-1B-PanIN-3 lesions (FIGS. 3 A, B).

### Immunohistochemical Expression Profile of AGR 2

Staining of the tissue arrays with the AGR2 antibody displayed a primarily intra-cytoplasmic and less membrane-based expression pattern. The average scores for the AGR2 staining were as follows: normal pancreatic ducts 7.59 (standard deviation 3.5, range 2-12); PanIN-1A 10.97 (standard deviation 2.0, range 6-12); PanIN-1B 10.16 (standard deviation 2.6, range 3-12); PanIN-2 8.96 (standard deviation 2.9, range 3-12); PanIN-3 8.47 (standard deviation 3.3, range 3-12), and PDAC 6.53 (standard deviation 2.6, range 1-12) (FIG. 2). Comparing the various groups with each other, significant score differences were detected (Kruskal-Wallis test, p<0.001). Furthermore it was shown that AGR2 is significantly stronger expressed in PanIN-1A, PanIN-1B, PanIN-2, and PanIN-3 than in normal pancreatic ducts (Mann-Whitney U test, p=0.002). Compared to the PanIN lesions, the ADR2 expression in PDAC was significantly decreased (Mann-Whitney U test, p<0.001). The results of the proteome analysis corresponded to the immunohistochemical reactions for PanIN-1A-PanIN-3.

### Differential Diagnosis of Pancreatic Cancer, PDAC and Pancreatitis:

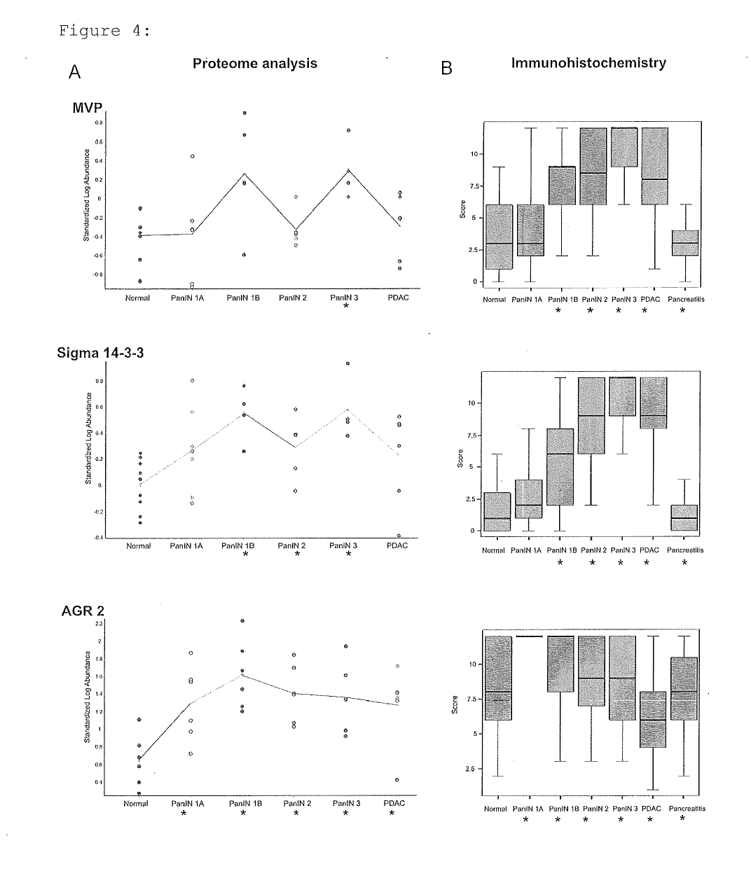

For the proteins AGR2, 14-3-3 sigma and MVP, the present study revealed increased expression during progression of PDAC by proteome investigation and by immunohistochemical analysis. In order to evaluate the application of these proteins to differentiate between pancreatic cancer and pancreatitis, their expression was also studied in tissue arrays of 40 pancreatitis patients. In contrast to pancreatic cancer patients, there was no or little detectable concentration in pancreatitis patients. The expression level of these proteins in the tissue of the pancreatitis patients is comparable to the level of expression in healthy tissue. Thus, AGR2, 14-3-3 sigma and MVP show a high potential for being used as non-invasive or in vivo biomarkers to differentiate (differential diagnosis) between PDAC or pancreatic cancer and pancreatitis (see FIG. 4).

The sequences according to the invention (SEQ ID No. 1-71) are as follows:

SEQ ID No. 1
>gi|33875698|gb|AAH00654.2| KRT8 protein
[Homo sapiens]
FSAPSRISAWFGPPASTPASTMSIRVIQKSYKVSTSGPRAFSS
RSYTSGPGSRISSSSFSRVGSSNERGGLGGGYGGASGMGGITA
VTVNQSLLSPLVLEVDPNIQAVRTQEKEQIKTLNNKFASFIDK
VRFLEQQNKMLETKWSLLQQQKTARSNMDNMFESYINNLRRQL
ETLGQEKLKLEAELGNMQGLVEDFKNKYEDEINKRTEMENEFV
LIKKDVDEAYMNKVELESRLEGLIDEINFLRQLYEEEIRELQS
QISDISVVLSMDNSRSLDMDSIIAEVKAQYEDIANRSRAEAES
MYQIKYEELQSLAGKHGDDLRRTKTEISEMNRNISRLQAEIEG
LKGQRASLEAAIADAEQRGELAIKDANAKLSELEAALQRAKQD
MARQLREYQELMNVKLALDIEIATYRKLLEGEESRLESGMQNM
SIHTKTTSGYAGGLSSAYGGLTSPGLSYSLGSSEGSGAGSSSF
SRTSSSRAVVVKKIETRDGKLVSESSDVLPK
SEQ ID No. 2
>gi|7576229|emb|CAB87963.1| vimentin
[Homo sapiens]
MSTRSVSSSSYRRMFGGPGTASRPSSSRSYVTTSTRTYSLGSA
LRPSTSRSLYASSPGGVYATRSSAVRLRSSVPGVRLLQDSVDF
SLADAINTEEKNTRTNEKVELQELNDRFANYIDKVRFLEQQNK
ILLAELEQLKGQGKSRLGDLYEEEMRELRRQVDQLINDKARVE
VERDNLAEDIMRLREKLQEEMLQREEAENTLQSFRQDVDNASL
ARLDLERKVESLQEEIAFLKKLHEEEIQELQAQIQEQHVQIDV
DVSKPDLTAALRDVRQQYESVAAKNLQEAEEWYKSKFADLSEA
ANRNNDALRQAKQESTEYRRQVQSLTCEVDALKGTNESLERQM
REMEENFAVEAANYQDTIGRLQDEIQNMKEEMARHLREYQDLL
NVKMALDIEIATYRKLLEGEESRISLPLPNFSSLNLRETNLDS
LPLVDTHSKRTLLIKTVETRDGQVINETSQHHDDLE
SEQ ID No. 3
>gi|12804929|gb|AAH01917.1| Malate
dehydrogenase 2, NAD (mitochondrial)
[Homo sapiens]
MLSALARPVSAALRRSFSTSAQNNAKVAVLGASGGIGQPLSLL
LKNSPLVSRLTLYDIAHTPGVAADLSHIETKAAVKGYLGPEQL
PDCLKGCDVVVIPAGVPRKPGMTRDDLENTNATIVATLTAACA
QHCPEAMICVIANPVNSTIPITAEVEKKHGVYNPNKIFGVTTL
DIVRANTFVAELKGLDPARVNVPVIGGHAGKTIIPLISQCTPK
VDFPQDQLTALTGRIQEAGTEVVKAKAGAGSATLSMAYAGARF
VFSLVDAMNGKEGVVECSFVKSQETECTYFSTPLLLGKKGIEK
NLGIGKVSSFEEKMISDAIPELKASIKKGEDFVKTLK
SEQ ID No. 4
>gi|6573280|gb|AAF17621.1| beta tropomyosin
[Homo sapiens]
MDAIKKKMQMLKLDKENAIDRAEQAEADKKQAEDRCKQLEEEQ
QALQKKLKGTEDEVEKYSESVKEAQEKLEQAEKKATDAEADVA
SLNRRIQLVEEELDRAQERLATALQKLEEAEKAADESERGMKV
IENRAMKDEEKMELQEMQLKEAKHIAEDSDRKYEEVARKLVIL
EGELERSEERAEVAESRARQLEEELRTMDQALKSLMASEEEYS
TKEDKYEEEIKLLEEKLKEAETRAEFAERSVAKLEKTIDDLE
SEQ ID No. 5
>gi|40226101|gb|AAH23548.11 ACTG1 protein
[Homo sapiens]
KANREKMTQIMFETENTPAMYVAIQAVLSLYASGRTIGIVMDS
GDGVIHTVPIYEGYALPHAILRLDLAGRDLTDYLMKILTERGY
SFITTAEREIVRDIKEKLCYVALDFEQEMATAASSSSLEKSYE
LPDGQVITIGNERFRCPEALFQPSFLGMESCGIHETTENSIMK
CDVDIRKDLYANTVLSGGITMYPGIADRMQKEITALAPSTMKI
KIIAPPERKYSVWIGGSILASLSTFQQMWISKQEYDESGPSIV
HRKCF
SEQ ID No. 6
>gi|3153859|gb|AAC17430.1| thioredoxin
delta 3 [Homo sapiens]
VKQIESKTAFQEALDAAGDKLVVVDFSATWCGPCKMIKPFFHD
VASECEVKCMPTFQFFKKGQKVGEFSGANKEKLEATINELV
SEQ ID No. 7
>gi|999893|pdb|1HTI|B Chain B,
Triosephosphate Isomerase (Tim)
(E.C.5.3.1.1) Complexed With 2-Phospho-
glycolic Acid
APSRKFFVGGNWKMNGRKQSLGELIGILNAAKVPADTEVVCAP
PTAYIDFARQKLDPKIAVAAQNCYKVTNGAFTGEISPGMIKDC
GATWVVLGHSERRHVFGESDELIGQKVAHALAEGLGVIACIGE
KLDEREAGITEKVVFEQTKVIADNVKDWSKVVLAYEPVWAIGI
GKTATPQQAQEVHEKLRGWLKSNVSDAVAQSTRIIYGGSVTGA
TCKELASQPDVDGFLVGGASLKPEFVDIINAKQ
SEQ ID No. 8
>gi|16306978|gb|AAH09564.1| Annexin A2
[Homo sapiens]
MSTVHEILCKLSLEGDHSTPPSAYGSVKAYTNFDAERDALNIE
TAIKTKGVDEVTIVNILTNRSNAQRQDIAFAYQRRTKKELASA
LKSALSGHLETLILGLLKTPAQYDASELKASMKGLGTDEDSLI
EIICSRTNQELQEINRVYKEMYKTDLEKDIISDTSGDFRKLMV
ALAKGRRAEDGSVIDYELIDQDARDLYDAGVKRKGTDVPKWIS
IMTERSVPHLQKVFDRYKSYSPYDMLESIRKEVKGDLENAFLN
LVQCIQNKPLYFADRLYDSMKGKGTRDKVLIRIMVSRSEVDML
KIRSEFKRKYGKSLYYYIQQDTKGDYQKALLYLCGGDD
SEQ ID No. 9
>gi|10441386|gb|AAG17014.1|AF186109_1 TPM4-
ALK fusion oncoprotein type 2
[Homo sapiens]
MAGLNSLEAVKRKIQALQQQADEAEDRAQGLQRELDGERERRE
KAEGDVAALNRRIQLVEEELDRAQERLATALQKLEEAEKAADE
SERGMKVIENRAMKDEEKMEIQEMQLKEAKHIAEEADRKYEEV
ARKLVILEGELERAEERAEVSELKCGDLEEELKNVTNNLKSLE
AASEKYSEKEDKYEEEIKLLSDKLKEAETRAEFAERTVAKLEK
TIDDLEVYRRKHQELQAMQMEL
SEQ ID No. 10
>gi|62205349|gb|AAH93076.1| PPIA protein
[Homo sapiens]
MCQGGDFTRHNGTGGKSIYGEKFEDENFILKHTGPGILSMANA
GPNTNGSQFFICTAKTEWLDGKHVVFGKVKEGMNIVEAMERFG
SRNGKTSKKITIADCGQLE
SEQ ID No. 11
>gi|189022|gb|AAA36348.1| smooth muscle
mysoin light chain
MRALGQNPTNAEVEKVLGNPKSDEMNVKVLDFEHFLPMLQTVA
KNKDQGTYEDYVEGERVEDKEGNGTVMGAEIRHVLVTLGEKMT
EEEVEMLVAGHEDSNGCINYEAFVRHILSG
SEQ ID No. 12
>gi|1408188|gb|AAC50680.11 desmin
MSQAYSSSQRVSSYRRTFGGAPGFPLGSPLSSPVFPRAGFGSK
GSSSSVTSRVYQVSRTSGGAGGLGSLRASRLGTTRTPSSYGAG
ELLDFSLADAVNQEFLTTRTNEKVELQELNDRFANYIEKVRFL
EQQNALAAEVNRLKGREPTRVAELYEEELRELRRQVEVLTNQR
ARVDVERDNLLDDLQRLKAKLQEETQLKEEAENNLAAFRADVD
AATLARIDLERRIESLNEETAFLKKVHEEEIRELQAQLQEQQV
QVEMDMSKPDLTAALRDIRAQYETIAAKNISEAEEWYKSKVSD
LTQAANKNNDALRQAKQEMMEYRHQIQSYTCEIDALKGTNDSL
MRQMRELEDRFASEASGYQDNIARLEEEIRHLKDEMARHLREY
QDLENVKMALDVEIATYRKLLEGEESRINLPIQTYSALNFRET
SPEQRGSEVHTKKTVMIKTIETRDGEVVSEATQQQHEVL
SEQ ID No. 13
>gi|15990478|gb|AAH15623.1| Major vault
protein [Homo sapiens]
MATEEFIIRIPPYHYIHVLDQNSNVSRVEVGPKTYIRQDNERV
LFAPMRMVTVPPRHYCTVANPVSRDAQGLVLFDVTGQVRLRHA
DLEIRLAQDPFPLYPGEVLEKDITPLQVVLPNTALHLKALLDF
EDKDGDKVVAGDEWLFEGPGTYIPRKEVEVVEITQATIIRQNQ
ALRLRARKECWDRDGKERVTGEEWLVTTVGAYLPAVFEEVLDL
VDAVILTEKTALHLRARRNFRDFRGVSRRTGEEWLVTVQDTEA
HVPDVHEEVLGVVPITTLGPHNYCVILDPVGPDGKNQLGQKRV
VKGEKSFFLQPGEQLEQGIQDVYVLSEQQGLLLRALQPLEEGE
DEEKVSHQAGDHWLIRGPLEYVPSAKVEVVEERQAIPLDENEG
IYVQDVKTGKVRAVIGSTYMETQDEVLWEKELPPGVEELLNKG
QDPLADRGEKDTAKSLQPLAPRNKTRVVSYRVPHNAAVQVYDY
REKRARVVEGPELVSLGPEEQFTVLSLSAGRPKRPHARRALCL
LLGPDFFTDVITIETADHARLQLQLAYNWHFEVNDRKDPQETA
KLFSVPDFVGDACKAIASRVRGAVASVTFDDFHKNSARIIRTA
VEGFETSEAKGPDGMALPRPRDQAVFPQNGLVVSSVDVQSVEP
VDQRTRDALQRSVQLAIEITTNSQEAAAKHEAQRLEQEARGRL
ERQKILDQSEAEKARKELLELEALSMAVESTGTAKAEAESRAE
AARIEGEGSVLQAKLKAQALAIETEAELQRVQKVRELELVYAR
AQLELEVSKAQQLAEVEVKKFKQMTEAIGPSTIRDLAVAGPEM
QVKLLQSLGLKSTLITDGSTPINLENTAFGLLGMGPEGQPLGR
RVASGPSPGEGISPQSAQAPQAPGDNHVVPVLR
SEQ ID No. 14
>gi|14043070|ref|NP_112420.1| heterogeneous
nuclear ribonucleoprotein A1 isoform b
[Homo sapiens]
MSKSESPKEPEQLRKLFIGGLSFETTDESLRSHFEQWGTLTDC
VVMRDPNTKRSRGEGFVTYATVEEVDAAMNARPHKVDGRVVEP
KRAVSREDSQRPGAHLTVKKIFVGGIKEDTEEHHLRDYFEQYG
KIEVIEIMTDRGSGKKRGFAFVTFDDHDSVDKIVIQKYHTVNG
HNCEVRKALSKQEMASASSSQRGRSGSGNFGGGRGGGFGGNDN
FGRGGNFSGRGGFGGSRGGGGYGGSGDGYNGFGNDGGYGGGGP
GYSGGSRGYGSGGQGYGNQGSGYGGSGSYDSYNNGGGGGFGGG
SGSNFGGGGSYNDFGNYNNQSSNFGPMKGGNFGGRSSGPYGGG
GQYFAKPRNQGGYGGSSSSSSYGSGRRF
SEQ ID No. 15
>gi|4388970|pdb|1BT6|B Chain B, P11
(S100a10), Ligand Of Annexin Ii In Complex
With Annexin Ii N-Terminus
PSQMEHAMETMMFTFHKFAGDKGYLTKEDLRVLMEKEFPGFLE
NQKDPLAVDKIMKDLDQCRDGKVGFQSFFSLIAGLTIACNDYF
VVHMKQKGKK
SEQ ID No. 16
>gi|24210508|gb|AAN51932.1|AF322220_1
cervical cancer suppressor 3 (EF1a-like
protein
MITGTSQADCAVLIVAAGVGEFEAGISKNGQTREHALLAYTEG
VKQLIVGVNKMDSTEPPYSQKRYEEIVKEVSTYIKKIGYNPDT
VAFVPISGWNGDNMLEPSANMPWFKGWKVTRKDGNASGTTLLE
ALDCILPPTRPTDKPLREPLQDVYKIGGIGTVPVGRVETGVLK
PGMVVTFAPVNVTTEVKSVEMHHEALSEALPGDNVGENVKNVS
VKDVRRGNVAGDSKNDPPMEAAGETAQVIILNHPGQISAGYAP
VLDCHTAHIACKFAELKEKIDRRSGKKLEDGPKFLKSGDAAIV
DMVPGKPMCVESFSDYPPLGRFAVRDMRQTVAVGVIKAVDKKA
AGAGKVTKSAQKAQKAK
SEQ ID No. 17
>gi|33338062|gb|AAQ13653.1| regulatory
myosin light chain long version
[Homo sapiens]
MSSKRAKAKTTKKRPQRATSNVFAMFDQSQIQEFKEAFNMIDQ
NRDGFIDKEDLHDMLASLGKNPTDEYLEGMMSEAPGPYNFTMF
LTMFGEKLNGTDPEDVIRNAFACFDEESSGFIHEDHLRELLTT
MGDRFTDEEVDEMYREAPIDKKGNFNYVEFTRILKHGAKDKDD
SEQ ID No. 18
>gi|63252896|ref|NP_001018004.1|
tropomyosin 1 alpha chain isoform 3
[Homo sapiens]
MDAIKKKMQMLKLDKENALDRAEQAEADKKAAEDRSKQLEDEL
VSLQKKLKGTEDELDKYSEALKDAQEKLELAEKKATDAEADVA
SLNRRIQLVEEELDRAQERLATALQKLEEAEKAADESERGMKV
IESRAQKDEEKMEIQEIQLKEAKHIAEDADRKYEEVARKLVII
ESDLERAEERAELSEGKCAELEEELKTVTNNLKSLEAQAEKYS
QKEDRYEEEIKVLSDKLKEAETRAEFAERSVTKLEKSIDDLEE
KVAHAKEENLSMHQMLDQTLLELNNM
SEQ ID No. 19
>gi|55859703|emb|CAI10974.1| tropomyosin 2
(beta) [Homo sapiens]
MDAIKKKMQMLKLDKENAIDRAEQAEADKKQAEDRCKQLEEEQ
QALQKKLKGTEDEVEKYSESVKEAQEKLEQAEKKATDAEADVA
SLNRRIQLVEEELDRAQERLATALQKLEEAEKAADESERGMKV
IENRAMKDEEKMELQEMQLKEAKHIAEDSDRKYEEVARKLVIL
EGELERSEERAEVAESRARQLEEELRTMDQALKSLMASEEEYS
TKEDKYEEEINLLEEKLKEAETRAEFAERSVAKLEKTIDDLEE
TLASAKEENVEIHQTLDQTLLELNNL
SEQ ID No. 20
>gi|62896697|dbj|BAD96289.1| myosin
regulatory light chain MRCL3 variant
[Homo sapiens]
MSSKRTKTKTKKRPQRATSNVFAMFDQSQIQEFKEAFNMIDQN
RNGFIDKEDLHDMLASLGKNPTDEYLDAMMNEAPGPINFTMFL
TMFGEKLNGTDPEDVIRNAFACFDEEATGTIQEDYLRELLTTM
GDRFTDEEVDELYREAPIDKKGNFNYIEFTRILKHGAKDKDD
SEQ ID No. 21
>gi|1335076|emb|CAA23774.1| alpha-2-globin
[Homo sapiens]
VLSPADKTNVKAAWGKVGAHAGEYGAEALERMFLSFPTTKTYF
PHFDLSHGSAQVKGHGKKVADALTNAVAHVDDMPNALSALSDL
HAHKLRVDPVNFKLLSHCLLVTLAAHLPAEFTPAVHASLDKFL
ASVSTVLTSKYR
SEQ ID No. 22
>gi|12803959|gb|AAH02827.1| Tropomyosin 4
[Homo sapiens]
MAGLNSLEAVKRKIQALQQQADEAEDRAQGLQRELDGERERRE
KAEGDVAALNRRIQLFEEELDRAQERLATALQKLEEAEKAADE
SERGMKVIENRAMKDEEKMEIQEMQLKEAKHIAEEADRKYEEV
ARKLVILEGELERAEERAEVSELKCGDLEEELKNVTNNLKSLE
AASEKYSEKEDKYEEEIKLLSDKLKEAETRAEFAERTVAKLEK
TIDDLEEKLAQAKEENVGLHQTLDQTLNELNCI
SEQ ID No. 23
>gi|62205326|gb|AAH93050.1| Transgelin
[Homo sapiens]
MANKGPSYGMSREVQSKIEKKYDEELEERLVEWIIVQCGPDVG
RPDRGRLGFQVWLKNGVILSKLVNSLYPDGSKPVKVPENPPSM
VFKQMEQVAQFLKAAEDYGVIKTDMFQTVDLFEGKDMAAVQRT
LMALGSLAVTKNDGHYRGDPNWFMKKAQEHKREFTESQLQEGK
HVIGLQMGSNRGASQAGMTGYGRPRQIIS
SEQ ID No. 24
>gi|60655723|gb|AAX32425.1| keratin 7
[synthetic construct]
MSIHFSSPVFTSRSAAFSGRGAQVRLSSARPGGLGSSSLYGLG
ASRPRVAVRSAYGGPVGAGIREVTINQSLLAPLRLDADPSLQR
VRQEESEQIKTLNNKFASFIDKVRFLEQQNKLLETKWTLLQEQ
KSAKSSRLPDIFEAQIAGLRGQLEALQVDGGRLEAELRSMQDV
VEDEKNKYEDEINRRTAAENEFVVLKKDVDAAYMSKVELEAKV
DALNDEINFLRTLNETELTELQSQISDTSVVLSMDNSRSLDLD
GIIAEVKAQYEEMAKCSRAEAEAWYQTKFETLQAQAGKHGDDL
RNTRNEISEMNRAIQRLQAEIDNIKNQRAKLEAAIAEAEERGE
LALKDARAKQEELEAALQRAKQDMARQLREYQELMSVKLALDI
EIATYRKLLEGEESRLAGDGVGAVNISVMNSTGGSSSGGGIGL
TLGGTMGSNALSFSSSAGPGLLKAYSIRTASASRRSARD
SEQ ID No. 25
>gi|15277503|gb|AAH12854.1| ACTB protein
[Homo sapiens]
MCKAGFAGDDAPRAVEPSIVGRPRHQGVMVGMGQKDSYVGDEA
QSKRGILTLKYPIEHGIVTNWDDMEKIWHHTFYNELRVAPEEH
PVLLTEAPLNPKANLEKMTQIMFETENTPAMYVAIQAVLSLYA
SGRTTGIVMDSGDGVIHTVPIYEGYALPHAILRLDLAGRDLTD
YLMKILTERGYSETTTAEREIVRDIKEKLCYVALDEEQEMATA
ASSSSLEKSYELPDGQVITIGNERFRCPEALFQPSFLGMESCG
IHETTENSIMKCDVDIRKDLYANTVLSGGTTMYPGIADRMQKE
ITALAPSTMKIKIIAPPERKYSVWIGGSILASLSTFQQMWISK
QEYDESGPSIVHRKCF
SEQ ID No. 26
>gi|33286422|ref|NP_872271.1| pyruvate
kinase 3 isoform 2 [Homo sapiens]
MSKPHSEAGTAFIQTQQLHAAMADTFLEHMCRLDIDSPPITAR
NTGIICTIGPASRSVETLKEMIKSGMNVARLNFSHGTHEYHAE
TIKNVRTATESFASDPILYRPVAVALDTKGPEIRTGLIKGSGT
AEVELKKGATLKITLDNAYMEKCDENILWLDYKNICKVVEVGS
KIYVDDGLISLQVKQKGADFLVTEVENGGSLGSKKGVNLPGAA
VDLPAVSEKDIQDLKFGVEQDVDMVFASFIRKASDVHEVRKVL
GEKGKNIKIISKIENHEGVRRFDEILEASDGIMVARGDLGIEI
PAEKVFLAQKMMIGRCNRAGKPVICATQMLESMIKKPRPTRAE
GSDVANAVLDGADCIMLSGETAKGDYPLEAVRMQHLIAREAEA
AMEHRKLFEELVRASSHSTDLMEAMAMGSVEASYKCLAAALIV
LTESGRSAHQVARYRPRAPIIAVTRNPQTARQAHLYRGIFPVL
CKDPVQEAWAEDVDLRVNFAMNVGKARGFFKKGDVVIVLTGWR
PGSGFTNTMRVVPVP
SEQ ID No. 27
>gi|56204524|emb|CAI19482.1| actin related
protein 2/3 complex, subunit 5, 16 kDa
[Homo sapiens]
MSKNIVSSARFRKVDVDEYDENKFVDEEDGGDGQAGPDEGEVD
SCLRHSITGNMTAALQAALKNPPINTKSQAVKDRAGSIVLKVL
ISFKANDIEKAVQSLDKNGVDLLMKYIYKGFESPSDNSSAMLL
QWHEKALAAGGVGSIVRVLTARKTV
SEQ ID No. 28
>gi|37183136|gb|AAQ89368.1| AGR2
[Homo sapiens]
MEKIPVSAFLLLVALSYTLARDTTVKPGAKKDTKDSRPKLPQT
LSRGWGDQLIWTQTYEEALYKSKTSNKPLMIIHRLDECPHSQA
LKKVFAENKEIQKLAEQFVLLNLVYETTDKHLSPDGQYVPRIM
FVDPSLTVRADITGRYSNRLYAYEPADTALLLDNMKKALKLLK
TEL
SEQ ID No. 29
>gi|7981260|emb|CAB92118.1| stratifin
[Homo sapiens]
MERASLIQKAKLAEQAERYEDMAAFMKGAVEKGEELSCEERNL
LSVAYKNVVGGQRAAWRVLSSIEQKSNEEGSEEKGPEVREYRE
KVETELQGVCDTVLGLLDSHLIKEAGDAESRVFYLKMKGDYYR
YLAEVATGDDKKRIIDSARSAYQEAMDISKKEMPPTNPIRLGL
ALNESVEHYEIANSPEEAISLAKTTEDEAMADLHTLSEDSYKD
STLIMQLLRDNLTLWTADNAGEEGGEAPQEPQS
SEQ ID No. 30
>gi|27695621|gb|AAH42970.1| Coactosin-like
1 (Dictyostelium) [Homo sapiens]
MATKIDKEACRAAYNLVRDDGSAVIWVIFKYDGSTIVHGEQGAE
YQHFIQQCTDDVRLFAFVRFTTGDAMSKRSKFALITWIGENVSG
LQRAKTGIDKTLVKEVVQNFAKEEVISDRKELEEDFIKSELKKA
GGANYDAQTE
SEQ ID No. 31
>gi|6996447|emb|CAB75426.1| chaperonin 60,
Hsp60 [Homo sapiens]
MLRLPTVFRQMRPVSRVLAPHLTRAYAKDVKFGADARALMLQGV
DLLADAVAVTMGPKGRIVIIEQSWGSPKVIKDGVTVAKSIDLKD
KYKNIGAKLVQDVANNTNEEAGDGTITATVLARSIAKEGFEKIS
KGANPVEIRRGVMLAVDAVIAELKKQSKPVTIPEEIAQVATISA
NGDKEIGNIISDAMKKVGRKGVITVKDGKILNDELEIIEGMKFD
RGYISPYFINTSKGQKCEEQDAYVLLSEKKISSINIVPALEIAN
AHRKPLVIIAEDVDGEALSTLVLNRLKVGLQVVAVKAPGFGDNR
KNQLKDMAIATGGAVFGEEGLTLNLEDVQPHDLGKVGEVIVTKD
DAMLLKGKGDKAQIEKRIQEIIEQLDVTISEYEKEKLNERLAKL
SDGVAVLKVGGISDVEVNEKKDRVTDALNATRAAVEEGIVLGGG
CALLRCIPALDSLTPANEDQKIGIEIIKRTLKIPAMTIAKNAGV
EGSLIVEKIMQSSSEVGYDAMAGDFVNMVEKGIIDPIKVVRTAL
LDAAGVASLLTTAEVVVTEIPKEEKDPGMGAMGGMGGGMGGGMF
SEQ ID No. 32
>gi|55960373|emb|CAI14602.1| transgelin 2
[Homo sapiens]
MANRGPAYGLSREVQQKIEKQYDADLEQILIQWITTQCRKDVGR
PQPGRENFQNWLKDGTVLCELINALYPEGQAPVKKIQASTMAFK
QMEQISQFLQAAERYGINTTDIFQTVDLWEGKNMACVQRTLMNL
GGLAVARDDGLFSGDPNWFPKKSKENPRNFSDNQLQEGKNVIGL
QMGTNRGASQAGMTGYGMPRQIL
SEQ ID No. 33
>gi|2183299|gb|AAC51652.1| aldehyde
dehydrogenase 1 [Homo sapiens]
MSSSGTPDLPVLLTDLKIQYTKIFINNEWHDSVSGKKETVENPA
TEEELCQVEEGDKEDVDKAVKAARQAFQIGSPWRIMDASERGRL
LYKLADLIERDRLLLATMESMNGGKLYSNAYLSDLAGCIKTLRY
CAGWADKIQGRTIPIDGNEFTYTRHEPIGVCGQIIPWNFPLVML
IWKIGPALSCGNTVVVKPAEQTPLTALHVASLIKEAGEPPGVVN
IVPGYGPTAGAAISSHMDIDKVAFTGSTEVGKLIKEAAGKSNLK
RVTLELGGKSPCIVLADADLDNAVEFAHHGVEYHQGQCCIAASR
IFVEESIYDEFVRRSVERAKKYILGNPLTPGVTQGPQIDKEQYD
KILDLIESGKKEGAKLECGGGPWGNKGYFVQPTVESNVIDEMRI
AKEEIFGPVQQIMKEKSLDDVIKRANNTFYGLSAGVFTKDIDKA
ITISSALQAGTVWVNCYGVVSAQCPFGGFKMSGNGRELGEYGFH
EYTEVKTVTVKISQKNS
SEQ ID No. 34
>gi|49660012|gb|AAT68294.1| sarcomeric
tropomyosin kappa; TPM1-kappa [Homo sapiens]
MDAIKKKMQMLKLDKENALDRAEQAEADKKAAEDRSKQLEEDIA
AKEKLLRVSEDERDRVLEELHKAEDSLLAAEEAAAKAEADVASL
NRRIQLVEEELDRAQERLATALQKLEEAEKAADESERGMKVIES
RAQKDEEKMEIQEIQLKEAKHIAEDADRKYEEVARKLVIIESDL
ERAEERAELSEGKCAELEEELKTVTNDLKSLEAQAEKYSQKEDR
YEEEIKVLSDKLKEAETRAEFAERSVTKLEKSIDDLEDELYAQK
LKYKAISEELDHALNDMTSI
SEQ ID No. 35
>gi|12654115|gb|AAH00871.1| Annexin A3
[Homo sapiens]
MASIWVGHRGTVRDYPDFSPSVDAEAIQKAIRGIGTDEKMLISI
LTERSNAQRQLIVKEYQAAYGKELKDDLKGDLSGHFEHLMVALV
TPPAVFDAKQLKKSMKGAGTNEDALIEILTTRTSRQMKDISQAY
YTVYKKSLGDDISSETSGDFRKALLTLADGRRDESLKVDEHLAK
QDAQILYKAGENRWGTDEDKFTEILCLRSFPQLKLTFDEYRNIS
QKDIVDSIKGELSGHFEDLLLAIVNCVRNTPAFLAERLHRALKG
IGTDEFTLNRIMVSRSEIDLLDIRTEFKKHYGYSLYSAIKSDTS
GDYEITLLKICGGDD
SEQ ID No. 36
>gi|18462107|gb|AAL72118.1| delta-globin
[Homo sapiens]
MVHLTPEEKTAVNALWGKVNVDAVGGEALGRLLVVYPWTQRFFE
SFGDLSSPDAVMGNPKVKAHGKKVLGAFSDGLAHLDNLKGTESQ
LSELHCDKLHVDPENFRLLGNVLVCVLARNEGKEFTPQMQAAYQ
KVVAGVANALAHKYH
SEQ ID No. 37
>gi|28592|emb|CAA23754.1| serum albumin
[Homo sapiens]
MKWVTFISLLFLFSSAYSRGVERRDAHKSEVAHRFKDLGEENFK
ALVLIAFAQYLQQCPFEDHVKLVNEVTEFAKTCVADESAENCDK
SLHTLFGDKLCTVATLRETYGEMADCCAKQEPERNECFLQHKDD
NPNLPRLVRPEVDVMCTAFHDNEETFLKKYLYEIARRHPYFYAP
ELLFFAKRYKAAFTECCQAADKAACLLPKLDELRDEGKASSAKQ
RLKCASLQKFGERAFKAWAVARLSQRFPKAEFAEVSKLVTDLTK
VHTECCHGDLLECADDRADLAKYICENQDSISSKLKECCEKPLL
EKSHCIAEVENDEMPADLPSLAADFVESKDVCKNYAEAKDVFLG
MFLYEYARRHPDYSVVLLLRLAKTYETTLEKCCAAADPHECYAK
VEDEEKPLVEEPQNLIKQNCELFKQLGEYKFQNALLVRYTKKVP
QVSTPTLVEVSRNLGKVGSKCCKHPEAKRMPCAEDYLSVVLNQL
CVLHEKTPVSDRVTKCCTESLVNRRPCFSALEVDETYVPKEFNA
ETFTFHADICTLSEKERQIKKQTALVELVKHKPKATKEQLKAVM
DDFAAFVEKCCKADDKETCFAEEGKKLVAASQAALGL
SEQ ID No. 38
>gi|189617|gb|AAC41689.1| protein PP4-X
MAMATKGGTVKAASGENAMEDAQTLRKAMKGLGTDEDAIISVLA
YRNTAQRQEIRTAYKSTIGRDLIDDLKSELSGNFEQVIVGMMTP
TVLYDVQELQRAMKGAGTDEGCLIEILASRTPEEIRRISQTYQQ
QYGRSLEDDIRSDTSFMFQRVLVSLSAGGRDEGNYLDDALVRQD
AQDLYEAGEKKWGTDEVKFLTVLCSRNRNHLLHVFDEYKRISQK
DIEQSIKSETSGSFEDALLAIVKCMRNKSAYFAEKLYKSMKGLG
TDDNTLIRVMVSRAEIDMLDIRAHFKRLYGKSLYSFIKGDTSGD
YRKVLLVLCGGDD
SEQ ID No. 39
>gi|28634|emb|CAA32891.1| crystallin
[Homo sapiens]
MDVTIQHPWFKRTLGPFYPSRLFDQFFGEGLFEYDLLPFLSSTI
SPYYRQSLERTVLDSGISEVRSDRDKFVIFLDVKHFSPEDLTVK
VQDDFVEIHGKHNERQ
SEQ ID No. 40
>gi|2605594|dbj|BAA23323.1| myosin
regulatory light chain [Homo sapiens]
MSSKKAKTKTTKKRPQRATSNVFAMFDQSQIQEFKEAFNMIDQN
RDGFIDKEDLHDMLASLGKNPTDAYLDAMMNEAPGPINFTMFLT
MFGEKLNGTDPEDVIRNAFACFDEEATGTIQEDYLRELLTTMGD
RFTDEGVDELYREAPIDKKGNFNYIEFTRILKHGAKDKDD
SEQ ID No. 41
>gi|2183299|gb|AAC51652.1| aldehyde
dehydrogenase 1 [Homo sapiens]
MSSSGTPDLPVLLTDLKIQYTKIFINNEWHDSVSGKKFPVFNPA
TEEELCQVEEGDKEDVDKAVKAARQAFQIGSPWRTMDASERGRL
LYKLADLIERDRLLLATMESMNGGKLYSNAYLSDLAGCIKTLRY
CAGWADKIQGRTIPIDGNFFTYTRHEPIGVCGQIIPWNFPLVML
IWKIGPALSCGNTVVVKPAEQTPLTALHVASLIKEAGEPPGVVN
IVPGYGPTAGAAISSHMDIDKVAFTGSTEVGKLIKEAAGKSNLK
RVTLELGGKSPCIVLADADLDNAVEFAHHGVEYHQGQCCIAASR
IFVEESIYDEFVRRSVERAKKYILGNPLTPGVTQGPQIDKEQYD
KILDLIESGKKEGAKLECGGGPWGNKGYEVQPTVESNVITEMRI
AKEEIFGPVQQIMKEKSLDDVIKRANNTFYGLSAGVFTKDIDKA
ITISSALQAGTVWVNCYGVVSAQCPFGGFKMSGNGRELGEYGFH
EYTEVKTVTVKISQKNS
SEQ ID No. 42
>gi|21361176|ref|NP_000680.2| aldehyde
dehydrogenase 1A1 [Homo sapiens]
MSSSGTPDLPVLLTDLKIQYTKIFINNEWHDSVSGKKFTVENPA
TEEELCQVEEGDKEDVDKAVKAARQAFQIGSPWRTMDASERGRL
LYKLADLIERDRLLLATMESMNGGKLYSNAYLNDLAGCIKTLRY
CAGWADKIQGRTIPIDGNEFTYTRHEPIGVCGQIIPWNFPLVML
IWKIGPALSCGNTVVVKPAEQTPLTALHVASLIKEAGFPPGVVN
IVPGYGPTAGAAISSHMDIDKVAFTGSTEVGKLIKEAAGKSNLK
RVTLELGGKSPCIVLADADLDNAVEFAHHGVFYHQGQCCIAASR
IFVEESIYDEFVRRSVERAKKYILGNPLTPGVTQGPQIDKEQYD
KILDLIESGKKEGAKLECGGGPWGNKGYFVQPTVFSNVTDEMRI
AKEEIFGPVQQIMKFKSLDDVIKRANNTFYGLSAGVETKDIDKA
ITISSALQAGTVWVNCYGVVSAQCPEGGEKMSGNGRELGEYGEH
EYTEVKTVTVKISQKNS
SEQ ID No. 43
T-complex protein 1 subunit beta
ASLSLAPVNI FKAGADEERA ETARLTSFIG AIAIGDLVKS
TLGPKGMDKI LLSSGRDASL MVTNDGATIL KNIGVDNPAA
KVLVDMSRVQ DDEVGDGTTS VTVLAAELLR EAESLIAKKI
HPQTIIAGWR EATKAAREAL LSSAVDHGSD EVKFRQDLMN
IAGTTLSSKL LTHHKDHFTK LAVEAVLRLK GSGNLEAIHI
IKKLGGSLAD SYLDEGFLLD KKIGVNQPKR IENAKILIAN
TGMDTDKIKI FGSRVRVDST AKVAEIEHAE KEKMKEKVER
ILKHGINCFI NRQLIYNYPE QLFGAAGVMA IEHADFAGVE
RLALVTGGEI ASTFDHPELV KLGSCKLIEE VMIGEDKLIH
FSGVALGEAC TIVLRGATQQ ILDEAERSLH DALCVLAQTV
KDSRTVYGGG CSEMLMAHAV TQLANRTPGK EAVAMESYAK
ALRMLPTIIA DNAGYDSADL VAQLRAAHSE GNTTAGLDMR
EGTIGDMAIL GITESFQVKR QVLLSAAEAA EVILRVDNII
KAAPRKRVPD HHPC
SEQ ID No. 44
Apolipoprotein A-IV
mflkavvltl alvavagara evsadqvatv mwdyfsqlsn
nakeavehlq kseltqqlna lfqdklgevn tyagdlqkkl
vpfatelher lakdseklke eigkeleelr arllphanev
sqkigdnlre lqqrlepyad qlrtgvntqa eqlrrqldpl
aqrmervlre nadslqaslr phadelkaki dqnveelkgr
ltpyadefkv kidqtveelr rslapyaqdt geklnhgleg
ltfqmkknae elkarisasa eelrqrlapl aedvrgnlkg
nteglqksla elgghldqqv eefrrrvepy genfnkalvq
qmeglrqklg phagdveghl sflekdlrdk vnsffstfke
kesqdktlsl peleqqqeqg qeqqqeqvqm laples
SEQ ID No. 45
Malate dehydrogenase, mitochondrial
precursor
MLSALARPVS AALRRSFSTS AQNNAKVAVL GASGGIGQPL
SLLLKNSPLV SRLTLYDIAH TPGVAADLSH IETKAAVKGY
LGPEQLPDCL KGCDVVVIPA GVPRKPGMTR DDLFNTNATI
VATLTAACAQ HCPEAMICVI ANPVNSTIPI TAEVFKKHGV
YNPNKIFGVT TLDIVRANTF VAELKGLDPA RVNVPVIGGH
AGKTIIPLIS QCTPKVDFPQ DQLTALTGRI QEAGTEVVKA
KAGAGSATLS MAYAGARFVF SLVDAMNGKE GVVECSFVKS
QETECTYFST PLLLGKKGIE KNLGIGKVSS FEEKMISDAI
PELKASIKKG EDFVKTLK
SEQ ID No. 46
Voltage-dependent anion-selective channel
protein 1
AVPPTYADLG KSARDVFTKG YGFGLIKLDL KTKSENGLEF
TSSGSANTET TKVTGSLETK YRWTEYGLTF TEKWNTDNTL
GTEITVEDQL ARGLKLTFDS SFSPNTGKKN AKIKTGYKRE
HINLGCDMDF DIAGPSIRGA LVLGYEGWLA GYQMNFETAK
SRVTQSNFAV GYKTDEFQLH TNVNDGTEFG GSIYQKVNKK
LETAVNLAWT AGNSNTRFGI AAKYQIDPDA CFSAKVNNSS
LIGLGYTQTL KPGIKLTLSA LLDGKNVNAG GHKLGLGLEF
QA
SEQ ID No. 47
>gi|31645|emb|CAA25833.1| glyceraldehyde-
3-phosphate dehydrogenase [Homo sapiens]
MGKVKVGVNGFGRIGRLVTRAAFNSGKVDIVAINDPFIDLNYMV
YMFQYDSTHGKFHGTVKAENGKLVINGNPITIFQERDPSKIKWG
DAGAEYVVESTGVFTTMEKAGAHLQGGAKRVIISAPSADAPMFV
MGVNHEKYDNSLKIISNASCTTNCLAPLAKVIHDNEGIVEGLMT
TVHAITATQKTVDGPSGKLWRDGRGALQNIIPASTGAAKAVGKV
IPELDGKLTGMAFRVPTANVSVVDLTCRLEKPAKYDDIKKVVKQ
ASEGPLKGILGYTEHQVVSSDENSDTHSSTFDAGAGIALNDHFV
KLISWYDNEFGYSNRVVDLMAHMASKE
SEQ ID No. 48
>gi|35053|emb|CAA37794.1| uracil DNA
glycosylase [Homo sapiens]
MGKVKVGVNGFGRIGRLVTRAAFNSGKVDIVAINDPFIDLNYMV
YMFQYDSTHGKFHGTVKAENGKLVINGNPITIFQERDPSKIKWG
DAGAEYVVESTGVETTMEKAGAHLQGGAKRVIISAPSADAPMFV
MGVNHEKYDNSLKIISNASCTTNCLAPLAKVIHDNFGIVEGLMT
TVHAITATQKTVDGPSGNCGVMAAGLSRTSSLPLLALKAVGKVI
PELNGKLTGMAFRVPTANVSVVDLTCRLEKPAKYDDIKKVVKQA
SEGPLKGILGYTEHQVVSSDENSDTHSSTFDAGAGIALNDHFVK
LISWYDNEFGYSNRVVDLMASKE
SEQ ID No. 49
>gi|54303910|gb|AAV33305.1| aging-associated
gene 9 protein [Homo sapiens]
MGKVKVGVNGFGRIGRLVTRAAFNSGKVDIVAINDPFIDLNYMV
YMFQYDSTHGKEHGTVKAENGKLVINGNPITIFQERDPSKIKWG
DAGAEYVVESTGVETTMEKAGAHLQGGAKRVIISTPSADAPMLV
MGVNHEKYDNSLKIISNASCTTNCLAPLAKVIHDNFGIVEGLMT
TVHAITATQKTVDGPSGKLWRDGRGALQNIIPASTGAAKAVGKV
IPELNGKLTGMAFRVPTANVSVVDLTCRLEKPAKYDDIKKVVKQ
ASEGPLKGILGYTEHQVVSSDFNSDTHSSTFDAGAGIALNDHFV
KLISWYDNEFGYSNRVVDLMAHMASKE
SEQ ID No. 50
>gi|13543557|gb|AAH05935.1| Nipsnap homolog
3A (C. elegans) [Homo sapiens]
MLVLRSALTRALASRTLAPQMCSSFATGPRQYDGIFYEFRSYYL
KPSKMNEFLENFEKNAHLRTAHSELVGYWSVEFGGRMNTVFHIW
KYDNFAHRTEVRKALAKDKEWQEQFLIPNLALIDKQESEITYLV
PWCKLEKPPKEGVYELATFQMKPGGPALWGDAFKRAVHAHVNLG
YTKLVGVFHTEYGALNRVHVLWWNESADSRAAGRHKSHEDPRVV
AAVRESVNYLVSQQNMLLIPTSFSPLK
SEQ ID No. 51
>gi|33188452|ref|NP_859427.1| peroxiredoxin
2 isoform b [Homo sapiens]
MASGNARIGKPAPDFKATAVVDGAFKEVKLSDYKGINTPRKEGG
LGPLNIPLLADVTRRLSEDYGVLKTDEGIAYRGLFIIDGKGVLR
QITVNDLPVGRSVDEALRLVQAFQYTDEHGEVCPAGWKPGSDTI
KPNVDDSKEYFSKHN
SEQ ID No. 52
>gi|438069|emb|CAA80269.1| thiol-specific
antioxidant protein [Homo sapiens]
MASGNARIGKPAPDFKATAVVDGAFKEVKLSDYKGKYVVLFFYP
LDFTFVCPTETIAFTTVKRTSAKLGCEVLGVSVDSQFTHLAWIN
TPRKEGGLGPLNIPLLADVTRRLSEDYGVLKNDEGIAYRGLFII
DGKGVLRQITVNDLPVGRSVDEALRLVQAFQYTDEHGEVCPAAW
KPGRDTIKPNVDDSKEYFSKHN
SEQ ID No. 53
>gi|440308|gb|AAA50465.1| enhancer protein
MASGNARIGKPAPDFKATAVVDGAFKEVKLSDYKGKYVVLFFYP
LDFTFVCPTEITAFSNRAEDFRKLGCEVLGVSVDSQFNHLAWIN
TPRKEGGLGPLNIPLLGDVTRRLSEDYGVLKTDEGIAYRGLFII
DGKGVLRQITVNDLPVGRSVDEALRLVQAFQYTDEHGEVCPAGW
KPGSDTIKPNVDDSKEYFSKHN
SEQ ID No. 54
>gi|16198390|gb|AAH15848.1| Chromosome 17
open reading frame 25 [Homo sapiens]
MAARRALHFVFKVGNRFQTARFYRDVLGMKVLRHEEFEEGCKAA
CNGPYDGKWSKTMVGFGPEDDHFVAELTYNYGVGDYKLGNDFMG
ITLASSQAVSNARKLEWPLTEVAEGVFETEAPGGYKFYLQNRSL
PQSDPVLKVTLAVSDLQKSLNYWCNLLGMKIYEKDEEKQRALLG
YADNQCKLELQGVKGGVDHAAAFGRIAFSCPQKELPDLEDLMKR
ENQKILTPLVSLDTPGKATVQVVILADPDGHEICFVGDEAFREL
SKIDPEGSKLLDDAMAADKSDEWFAKHNKPKASG
SEQ ID No. 55
>gi|34850074|ref|NP_057164.21 hypothetical
protein LOC51031 [Homo sapiens]
MAARRALHFVFKVGNRFQTARFYRDVLGMKVLRHEEFEEGCKAA
CNGPYDGKWSKTMVGFGPEDDHFVAELTYNYGVGDYKLGNDFMG
ITLASSQAVSNARKLEWPLTEVAEGVFETEAPGGYKFYLQNRSL
PQSDPVLKVTLAVSDLQKSLNYWCNLLGMKIYEKDEEKQRALLG
YADNQCKLELQGVKGGVDHAAAFGRIAFSCPQKELPDLEDLMKR
ENQKILTPLVSLDTPGKATVQVVILADPDGHEICFVGDEAFREL
SKMDPEGSKLLDDAMSADKSDEWFAKHNKPKASG
SEQ ID No. 56
>gi|4929769|gb|AAD34145.1|AF151908 1 CGI-150
protein [Homo sapiens]
MRLTPFSLSTGNSFRYSRRLKKNIFGTAPALRVSEMSLRPSSRI
FPCFSRNGLDFTIVITLAQPPVPGISFIVAKPRLFPGAGSAGCG
LLERLFLSLLLGTGLRWCLRGCFPGARFCSTTSPEGHTTFTGLR
RSARTQRLAQGPKPGPPAATVARQTSRVSPAPPCSLRPGLRHES
APSGIGDVTARGALRGLGCTVRVTAACGGNHGCSQMLHFVFKVG
NRFQTARFYRDVLGMKVLRHEEFEEGCKAACNGPYDGKWSKTMV
GFGPEDDHFVAELTYNYGVGDYKLGNDFMGITLASSQAVSNARK
LEWPLTEVAEGVFETEAPGGYKFYLQNRSLPQSDPVLKVTLAVS
DLQKSLNYWCNLLGMKIYEKDEEKQRALLGYADNQCKLELQGVK
GGVDHAAAFGRIAFSCPQKELPDLEDLMKRENQKILTPLVSLDT
PGKATVQVVILADPDGHEICFVGDEAFRELSKMDPEGSKLLDDA
MAADKSDEWFAKHNKPKASG
SEQ ID No. 57
>gi|19684181|gb|AAH26033.1| Gelsolin
(amyloidosis, Finnish type) [Homo sapiens]
MAPHRPAPALLCALSLALCALSLPVRAATASRGASQAGAPQGRV
PEARPNSMVVEHPEFLKAGKEPGLQIWRVEKFDLVPVPTNLYGD
FFTGDAYVILKTVQLRNGNLQYDLHYWLGNECSQDESGAAAIFT
VQLDDYLNGRAVQHREVQGFESATFLGYFKSGLKYKKGGVASGF
KHVVPNEVVVQRLFQVKGRRVVRATEVPVSWESFNNGDCFILDL
GNNIHQWCGSNSNRYERLKATQVSKGIRDNERSGRARVHVSEEG
TEPEAMLQVLGPKPALPAGTEDTAKEDAANRKLAKLYKVSNGAG
TMSVSLVADENPFAQGALKSEDCFILDHGKDGKIFVWKGKQANT
EERKAALKTASDFITKMDYPKQTQVSVLPEGGETPLFKQFFKNW
RDPDQTDGLGLSYLSSHIANVERVPFDAATLHTSTAMAAQHGMD
DDGTGQKQIWRIEGSNKVPVDPATYGQFYGGDSYTILYNYRHGG
RQGQIIYNWQGAQSTQDEVAASAILTAQLDEELGGTPVQSRVVQ
GKEPAHLMSLFGGKPMITYKGGTSREGGQTAPASTRLFQVRANS
AGATRAVEVLPKAGALNSNDAFVLKTPSAAYLWVGTGASEAEKT
GAQELLRVLRAQPVQVAEGSEPDGFWEALGGKAAYRTSPRLKDK
KMDAHPPRLFACSNKIGREVIEEVPGELMQEDLATDDVMLLDTW
DQVFVWVGKDSQEEEKTEALTSAKRYIETDPANRDRRTPITVVK
QGFEPPSFVGWELGWDDDYWSVDPLDRAMAELAA
SEQ ID No. 58
Gelsolin precursor
MAPHRPAPAL LCALSLALCA LSLPVRAATA SRGASQAGAP
QGRVPEARPN SMVVEHPEFL KAGKEPGLQI WRVEKFDLVP
VPTNLYGDFF TGDAYVILKT VQLRNGNLQY DLHYWLGNEC
SQDESGAAAI FTVQLDDYLN GRAVQHREVQ GFESATFLGY
FKSGLKYKKG GVASGFKHVV PNEVVVQRLF QVKGRRVVRA
TEVPVSWESF NNGDCFILDL GNNIHQWCGS NSNRYERLKA
TQVSKGIRDN ERSGRARVHV SEEGTEPEAM LQVLGPKPAL
PAGTEDTAKE DAANRKLAKL YKVSNGAGTM SVSLVADENP
FAQGALKSED CFILDHGKDG KIFVWKGKQA NTEERKAALK
TASDFITKMD YPKQTQVSVL PEGGETPLFK QFFKNWRDPD
QTDGLGLSYL SSHIANVERV PFDAATLHTS TAMAAQHGMD
DDGTGQKQIW RIEGSNKVPV DPATYGQFYG GDSYIILYNY
RHGGRQGQII YNWQGAQSTQ DEVAASAILT AQLDEELGGT
PVQSRVVQGK EPAHLMSLFG GKPMIIYKGG TSREGGQTAP
ASTRLFQVRA NSAGATRAVE VLPKAGALNS NDAFVLKTPS
AAYLWVGTGA SEAEKTGAQE LLRVLRAQPV QVAEGSEPDG
FWEALGGKAA YRTSPRLKDK KMDAHPPRLF ACSNKIGRFV
IEEVPGELMQ EDLATDDVML LDTWDQVFVW VGKDSQEEEK
TEALTSAKRY IETDPANRDR RTPITVVKQG FEPPSFVGWF
LGWDDDYWSV DPLDRAMAEL AA
SEQ ID No. 59
>gi|3766197|gb|AAC64396.1| ATP-specific
succinyl-CoA synthetase beta subunit
[Homo sapiens]
FNNHGLQVQQQQQRNLSLHEYMSMELLQEAGVSVPKGYVAKSP
DEAYAIAKKLGSKDVVIKAQVLAGGRGKGTFESGLKGGVKIVE
SPEEAKAVSSQMIGKKLETKQTGEKGRICNQVLVCERKYPRRE
YYFAITMERSFQGPVLIGSSHGGVNIEDVAAETPEATIKEPID
IEEGIKKEQALQLAQKMGEPPNIVESAAENMVKLYSLFLKYDA
TMIEINPMVEDSDGAVLCMDAKINFDSNSAYRQKKIFDLQDWT
QEDERDKDAAKANLNYIGLDGNIGCLVNGAGLAMATMDIIKLH
GGTPANFLDVGGGATVHQVTEAFKLITSDKKVLAILVNIEGGI
MRCDVIAQGIVMAVKDLEIKIPVVVRLQGTRVDDAKALIADSG
LKILACDDLDEAARMVVKLSEIVTLAKQAHVDVKFQLPI
SEQ ID No. 60
>gi|56204104|emb|CAI22099.1| TAR DNA
binding protein [Homo sapiens]
MSEYIRVTEDENDEPIEIPSEDDGTVLLSTVTAQFPGACGLRY
RNPVSQCMRGVRLVEGILHAPDAGWGNLVYVVNYPKDNKRKMD
ETDASSAVKVKRAVQKTSDLIVLGLPWKTTEQDLKEYFSTFGE
VLMVQVKKDLKTGHSKGFGFVRFTEYETQVKVMSQRHMIDGRW
CDCKLPNSKQSQDEPLRSRKVFVGRCTEDMTEDELREFFSQYG
DVMDVFIPKPFRAFAFVTFADDQIAQSLCGEDLIIKGISVHIS
NAEPKHNSNRQLERSGREGVHLISNVYGRSTSLKVVL
SEQ ID No. 61
2,4-dienoyl-CoA reductase, mitochondrial
precursor
MKLPARVFFT LGSRLPCGLA PRRFFSYGTK ILYQNTEALQ
SKFFSPLQKA MLPPNSFQGK VAFITGGGTG LGKGMTTLLS
SLGAQCVIAS RKMDVLKATA EQISSQTGNK VHAIQCDVRD
PDMVQNTVSE LIKVAGHPNI VINNAAGNFI SPTERLSPNA
WKTITDIVLN GTAFVTLEIG KQLIKAQKGA AFLSITTIYA
ETGSGFVVPS ASAKAGVEAM SKSLAAEWGK YGMRFNVIQP
GPIKTKGAFS RLDPTGTFEK EMIGRIPCGR LGTVEELANL
AAFLCSDYAS WINGAVIKFD GGEEVLISGE FNDLRKVTKE
QWDTIEELIR KTKGS
SEQ ID No. 62
>gi|49168580|emb|CAG38785.1| MDH2
[Homo sapiens]
MLSALVRPVSAALRRSFSTSAQNNAKVAVLGASGGIGQPLSLL
LKNSPLVSRLTLYDIAHTPGVAADLSHIETKAAVKGYLGPEQL
PDCLKGCDVVVIPAGVPRKPGMTRDDLENTNATIVATLTAACA
QHCPEAMICVIANPVNSTIPITAEVFKKHGVYNPNKIFGVTTL
DIVRANTFVAELKGLDPARVNVPVIGGHAGKTIIPLISQCTPK
VDFPQDQLTALTGRIQEAGTEVVKAKAGAGSATLSMAYAGARF
VFSLVDAMNGKEGVVECSFVKSQETECTYFSTPLLLGKKGIEK
NLGIGKVSSFEEKMISDAIPELKASIKKGEDFVKTLK
SEQ ID No. 63
Heat-shock protein beta-1
MTERRVPFSL LRGPSWDPFR DWYPHSRLFD QAFGLPRLPE
EWSQWLGGSS WPGYVRPLPP AAIESPAVAA PAYSRALSRQ
LSSGVSEIRH TADRWRVSLD VNHFAPDELT VKTKDGVVEI
TGKHEERQDE HGYISRCFTR KYTLPPGVDP TQVSSSLSPE
GTLTVEAPMP KLATQSNEIT IPVTFESRAQ LGGPEAAKSD
ETAAK
SEQ ID No. 64
>gi|21735621|ref|NP_005909.21 mitochondrial
malate dehydrogenase precursor
[Homo sapiens]
MLSALARPASAALRRSFSTSAQNNAKVAVLGASGGIGQPLSLL
LKNSPLVSRLTLYDIAHTPGVAADLSHIETKAAVKGYLGPEQL
PDCLKGCDVVVIPAGVPRKPGMTRDDLENTNATIVATLTAACA
QHCPEAMICVIANPVNSTIPITAEVFKKHGVYNPNKIFGVTTL
DIVRANTFVAELKGLDPARVNVPVIGGHAGKTIIPLISQCTPK
VDFPQDQLTALTGRIQEAGTEVVKAKAGAGSATLSMAYAGARF
VFSLVDAMNGKEGVVECSFVKSQETECTYFSTPLLLGKKGIEK
NLGIGKVSSFEEKMISDAIPELKASIKKGEDFVKTLK
SEQ ID No. 65
>gi|27753613|gb|AA022156.1|AF202897_1
prostate and colon associated protein
[Homo sapiens]
MDCREMDLYEDYQSPFDFDAGVNKSYLYLSPSGNSSPPGSPTL
QKFGLLRTDPVPEEGEDVAATISATETLSEEEQEELRRELAKV
EEEIQTLSQVLAAKEKHLAEIKRKLGINSLQELKQNIAKGWQD
VTATSAYKKTSETLSQAGQKASAAFSSVGSVITKKLEDVKNSP
TEKSFEEKVENLKSKVGGTKPAGGDFGEVLNSAANASATTTEP
LPEKTQESL
SEQ ID No. 66
>gi|3757661|emb|CAA76365.1| secretagogin
[Homo sapiens]
MDSSREPTLGRLDAAGFWQVWRRFDADEKGYIEEKELDAFFLH
MLMKLGTDDTVMKANLHKVKQQFMTTQDASKDGRIRMKELAGM
FLSEDENFLLLFRRENPLDSSVEFMQIWRKYDADSSGFISAAE
LRNFLRDLFLHHKKAISEAKLEEYTGTMMKIFDRNKDGRLDLN
DLARILALQENFLLQFKMDACSTEERKRDFEKIFAYYDVSKTG
ALEGPEVDGFVKDMMELVQPSISGVDLDKFREILLRHCDVNKD
GKIQKSELALCLGLKINP
SEQ ID No. 67
>gi|49457021|emb|CAG46831.1| TPD52
[Homo sapiens]
MDRGEQGLLRTDPVPEEGEDVAATISATETLSEEEQEELRREL
AKVEEEIQTLSQVLAAKEKHLAEIKRKLGINSLQELKQNIAKG
WQDVTATSAYKKTSETLSQAGQKASAAFSSVGSVITKKPEDVK
NSPTFKSFEEKVENLKSKVGGTKPAGGDFGEVLNSAANASATT
TEPLPEKTQESL
SEQ ID No. 68
>gi|54695758|gb|AAV38251.1| tumor protein
D52 [Homo sapiens]
MDRGEQGLLRTDPVPEEGEDVAATISATETLSEEEQEELRREL
AKVEEEIQTLSQVLAAKEKHLAEIKRKLGINSLQELKQNIAKG
WQDVTATSAYKKTSETLSQAGQKASAAFSSVGSVITKKLEDVK
NSPTEKSFEEKVENLKSKVGGTKPAGGDFGEVLNSAANASATT
TEPLPEKTQESL
SEQ ID No. 69
>gi|62898994|dbj|BAD97351.1| N8 protein
long isoform (Fragment) variant
[Homo sapiens]
RESPAEARRSSARRGGRSEPGRAAGGGAAEDTRRRAGDMDRGE
QGLLRTDPVPEEGEDVAATISATETLSEKEQEELRRELAKVEE
EIQTLSQVLAAKEKHLAEIKRKLGINSLQELKQNIAKGWQDVT
ATSAYKKTSETLSQAGQKASAAFSSVGSVITKKLEDVKNSPTF
KSFEEKVENLKSKVGGTKPAGGDFGEVLNSAANASATTTEPLP
EKTQESL
SEQ ID No. 70
>gi|70608174|ref|NP_001020424.1| tumor
protein D52 isoform 2 [Homo sapiens]
MDRGEQGLLRTDPVPEEGEDVAATISATETLSEEEQEELRREL
AKVEEEIQTLSQVLAAKEKHLAEIKRKLGINSLQELKQNIAKG
WQDVTATSAYKKTSETLSQAGQKASAAFSSVGSVITKKLEDVK
LQAFSHSFSIRSIQHSISMPAMRNSPTEKSFEEKVENLKSKVG
GTKPAGGDFGEVLNSAANASATTTEPLPEKTQESL
SEQ ID No. 71
>gi|4507645|ref|NP_000356.1|
triosephosphate isomerase 1 [Homo sapiens]
MAPSRKFFVGGNWKMNGRKQSLGELIGTLNAAKVPADTEVVCA
PPTAYIDFARQKLDPKIAVAAQNCYKVTNGAFTGEISPGMIKD
CGATWVVLGHSERRHVEGESDELIGQKVAHALAEGLGVIACIG
EKLDEREAGITEKVVFEQTKVIADNVKDWSKVVLAYEPVWAIG
TGKTATPQQAQEVHEKLRGWLKSNVSDAVAQSTRITYGGSVTG
ATCKELASQPDVDGFLVGGASLKPEFVDIINAKQ

1. Method for diagnosis of pancreatic cancer or precursor and/or concomitant illnesses thereof, comprising determining at least one biomarker selected from the group
a.) Keratin 8 protein (SEQ ID No. 1), Vimentin (SEQ ID No. 2), Mitochondrial malate dehydrogenase (SEQ ID No. 3), Beta tropomyosin (SEQ ID No. 4), ACTG1 protein (SEQ ID No. 5), Thioredoxin delta 3 (SEQ ID No. 6), B Chain B Triosephosphate Isomerase (SEQ ID No. 7), Annexin A2 (SEQ ID No. 8), TPM4-ALK fusion oncoprotein type 2 (SEQ ID No. 9), Peptidylprolyl isomerase A (SEQ ID No. 10), Smooth muscle mysoin light chain (SEQ ID No. 11), Desmin (SEQ ID No. 12), Major vault protein 1 (SEQ ID No. 13), Heterogeneous nuclear ribonucleoprotein A1 (SEQ ID No. 14), S100A10 (SEQ ID No. 15), EFla-like protein (SEQ ID No. 16), Regulatory myosin light chain long version (SEQ ID No. 17), Tropomyosin 1 alpha chain isoform 3 (SEQ ID No. 18), Tropomyosin 2 (beta) isoform 2 (SEQ ID No. 19), Myosin regulatory light chain MRCL3 (SEQ ID No, 20), Alpha-2-globin (SEQ ID No. 21), Tropomyosin 4 (SEQ ID No. 22), Transgelin (SEQ ID No. 23), Keratin 7 (SEQ ID No. 24), ACTB protein (SEQ ID No. 25), M2-type pyruvate kinase (SEQ ID No. 26), Actin related protein ⅔ complex subunit 5 (SEQ ID No. 27), Anterior gradient 2 homolog (AGR 2) (SEQ ID No. 28), Stratifin (14-3-3 sigma) (SEQ ID No. 29), Coactosin-like 1 (SEQ ID No. 30), Chaperonin heat shock 60 kD protein 1 (SEQ ID No. 31), Transgelin 2 (SEQ ID No. 32), Aldehyde dehydrogenase 1 (SEQ ID No. 33), Sarcomeric tropomyosin kappa (SEQ ID No. 34), Annexin A3 (SEQ ID No. 35), Delta-globin (SEQ ID No. 36), Serum albumin (SEQ ID No. 37), Protein PP4-X (Annexin A4) (SEQ ID No. 38), Crystallin (SEQ ID No. 39), Myosin regulatory light chain MRCL3 (SEQ ID No. 40),
or
group b.) aldehyde dehydrogenase 1 (SEQ ID No. 41), Aldehyde dehydrogenase 1A1 (SEQ ID No. 42), T-complex protein 1 subunit beta (SEQ ID No. 43), Apolipoprotein A4 (SEQ ID No. 44), Malate dehydrogenase mitochondrial precursor (SEQ ID No. 45), Voltage-dependent anion selective channel protein 1 (SEQ ID No. 46), glyceraldehydes-3-phosphate dehydrogenase (SEQ ID No. 47), uracil DNA glycosylase (SEQ ID No. 48), aging-associated-associated 9 protein (SEQ ID No. 49), Nipsnap homolog 3A (SEQ ID No. 50), peroxiredoxin 2 isoform b (SEQ ID No. 51), thiol-specific antioxidant protein (SEQ ID No. 52), enhancer protein (SEQ ID No. 53), Chromosome 17 open reading frame 25 (SEQ ID No. 54), hypothetical protein LOC51031 (SEQ ID No. 55), CGI-150 protein (SEQ ID No. 56), Gelsolin isoform a (SEQ ID No. 57), Gelsolin precursor (SEQ ID No. 58), ATP-specific succinyl-CoA synthetase beta subunit (SEQ ID No. 59), TAR DNA binding protein (SEQ ID No. 60), 2,4-dienoyl-CoA reductase mitochondrial precursor (SEQ ID No. 61), MDH2 (SEQ ID No. 62), heat shock protein beta-1 (SEQ ID No. 63), mitochondrial malate dehydrogenase precursor MDH-2 (SEQ ID No. 64), prostate and colon associated protein (SEQ ID No. 65), secretagogin (SEQ ID No. 66), TPD 52 (SEQ ID No. 67), tumor protein D52 (SEQ ID No. 68), N8 protein long isoform (Fragment) variant (SEQ ID No. 69), tumor protein D52 isoform 2 (SEQ ID No. 70), triosephosphate isomerase 1 (SEQ ID No. 71)
or fragments und partial peptides thereof in a patient to be examined.

2. Method for diagnosis of pancreatic cancer or precursor and/or concomitant illnesses thereof, according to Claim 1, characterized in that the method is an in-vitro diagnosis.

3. Method for diagnosis of pancreatic cancer or precursor and/or concomitant illnesses thereof, according to Claim 1, wherein the precursor and/or concomitant illnesses are PDAC (Pancreatic ductal adenocarcinoma), PanIN (pancreatic intraepithelial neoplasias), pancreatic lesions, CP (chronic pancreatitis), including endocrine pancreatic tumors.

4. Method for diagnosis of pancreatic cancer or precursor and/or concomitant illnesses thereof according to claim 1, characterized in that a combination of biomarkers according to claim 1 comprises at least Stratifin (14-3-3 sigma) (SEQ ID No. 29) and/or Vimentin (SEQ ID No. 2) and/or Major vault protein 1 (SEQ ID No. 13) and/or Anterior gradient 2 homolog (AGR 2) (SEQ ID No. 28), and/or S100A10 (SEQ ID No. 15) and/or EFla-like protein (SEQ ID No. 16) and/or Annexin A2 (SEQ ID No. 8) and/or Annexin A4 (SEQ ID No. 38).

5. Method for diagnosis of pancreatic cancer according to claim 1 for making clinical decisions, in particular for further treatment and therapy with medicaments.

6. Method for diagnosis of pancreatic cancer according to claim 1, characterized in that the diagnosis is made for prophylaxis, prognosis, differential diagnostic early detection and identification, severity assessment, and prognostic assessment in conjunction with therapy.

7. Method according to claim 1, characterized in that parallel or simultaneous determinations of the markers are carried out.

8. Method according to claim 1 characterized in that a 2D-Elektrophoresis is carried out with an isoelectric focusing in the first dimension and a SDS gel electrophoresis in the second dimension.

9. Method according to claim 1, characterized in that the determinations are carried out on at least one patient sample.

10. Method according to claim 1, characterized in that the determinations are carried out using a rapid test, in particular in single- or multi-parameter determinations.

11. Kit for diagnosis according to claim 1 comprising detection reagents and further adjuvants.

12. Diagnostic device for carrying out a method according to claim 1, particularly a protein biochip, array or assay.
